(19) United States
(12) Patent Application Publication
(10) Pub. No.: US 2007/0202216 A1
(43) Pub. Date: Aug. 30, 2007

(54) PHYTASE ACTIVE YEAST

(75) Inventors: Thomas Andlid, Goteborg (SE); Ann-Sofie Sandberg, Molndal (SE); Jenny Veide, Goteborg (SE)

Correspondence Address: GAUTHIER & CONNORS, LLP, 225 FRANKLIN STREET, SUITE 2300, BOSTON, MA, 02110, US

(73) Assignee: CHALMERS TECHNOLOGY LICENSING AB, Goteborg (SE)

(21) Appl. No.: 11/693,771

(22) Filed: Mar. 30, 2007

Related U.S. Application Data
(62) Division of application No. 10/228,785, filed on Aug. 26, 2002.

(30) Foreign Application Priority Data
Mar. 22, 2002 (SE) 0200911-6

(51) Int. Cl. A23L 1/28; C12N 1/19; C12N 15/74; C12N 9/16; C12P 7/02

(52) U.S. Cl. 426/62; 435/132; 435/196; 435/254.21; 435/483

(57) ABSTRACT

The present invention relates to a method for producing a modified Saccaromyces cerevisiea having improved phytase activity, such a Saccaromyces cerevisiae, use of such a modified strain, as well as phytase production, and inositol isomers derived from use of such a modified strain.

### PRIORITY DATA

This application is a divisional of U.S. patent application Ser. No. 10/228,785 filed on Aug. 26, 2002 which claims priority from Swedish Pat. Appln. No. 0200911-6, filed Mar. 22, 2002, all of which are incorporated herein in their entirety.

### BACKGROUND OF THE INVENTION

The present invention relates to a phytase active yeast, in particular a modified Saccharomyces cerevisiae used for fermentation purposes, fermented products after fermentation using said modified Saccharomyces cerevisiae, the use of phytase obtained using said strain, as well as derived inositol phosphate derivatives including myoinositol.

Iron deficiency is one of the most common nutrition disorders worldwide. In addition to affecting a large proportion of infants, children and women in the developing world, iron deficiency is the only nutrient deficiency of significant prevalence in all developed nations as well. Deficiency of iron and probably zinc are highly prevalent in developing countries, where the diet is based on cereals and legumes but also in vulnerable population groups, in industrialized countries, with high requirements such as women of fertile age, infants and adolescents. In developing countries iron deficiency, due to poor bioavailability, retards normal brain development in infants and effects the success of a pregnancy by increasing premature deliveries, as well as morbidity of mother and child at or around child-birth. Zinc deficiency prevents normal child growth and greatly weakens the immune system leading to more infections. The body requires a high amount of calcium during growth. The amount of bone in later years is determined by the starting amount (the peak bone mass) and it's subsequent loss. Both are directly relevant for the subsequent development of osteoporosis.

Mild to moderate iron deficiency is often not recognized, but nevertheless may affect poorly defined parameters such as normal vigour, physical and mental endurance, and quality of life.

Iron deficiency is caused primarily by chronic blood loss. Using approximate values, inescapable loss of iron in sweat and cellular desquamation amounts to 1-2 mg per day. This is readily balanced in men by the absorption of 10% of the average dietary intake of 10-15 mg daily. In women of fertile ages, however, menstrual bleeding adds another 1 mg to the daily loss, making it more difficult to achieve an adequate balance.

Several haematologic and biochemical tests are well established for screening or diagnosis of iron deficiency individuals as well as for population based assessment.

The amount of iron absorbed from the diet at any one time is dependent on three factors: the quantity of iron, the composition of the diet and the behavior of the mucosa of the upper small bowel. Variation in the bioavailability (the portion of total iron in the diet absorbed and utilized by the organism) of food iron are of greater importance for iron nutrition than is the amount of iron in the diet. The haem iron in meat, poultry and fish is easily absorbed whatever the dietary composition whereas non-heme iron is markedly influenced by other ingredients in the diet. A number of promoters and inhibitors of iron absorption have been identified. The bioavailability of the iron in any particular diet ultimately depends on the relative quantities of promoters and inhibitors of iron absorption present in that diet.

Although the element is the second most abundant metal in the earth's crust, its low solubility makes its acquisition for metabolic use a major challenge. Most environmental iron exists as insoluble salts. Gastric acidity assists the conversion to absorbable forms, but the efficiency of this process is limited. Many plants produce powerful chelators, such as the phytate (organic polyphosphate), which are potent inhibitors of iron absorption found in e.g., cereals and legumes.

Zinc is a component of more than 300 enzymes needed to repair wounds, maintain fertility in adults and growth in children, synthesize protein, help cells reproduce, preserve vision, boost immunity, and protect against free radicals, among other functions.

Zinc deficiency is common in individuals or populations whose diets are low in sources of readily bioavailable zinc, such as meat, and high in unrefined cereals that are rich in phytate, a diet common in the poorer areas of the world. Vegetarian or lacto-vegetarian diets overall result in high intake of phytate, which is accompanied by a greater risk of zinc deficiency. Zinc deficiency in human caused by nutrition was first described in adolescent boys and girls in Egypt and Iran in the late 1960:s and early 1970's. The symptoms were severe growth failure, hypogonadism with delayed sexual maturation. The cause of the deficiency was a diet based on unleavened wholemeal bread with a high content of phytate and low intake of animal protein. Treatment with zinc and an adequate intake of other nutrients improved growth and sexual maturation. Dietary zinc deficiency has later been described among the children of many countries, such as Turkey, China and Yugoslavia,. Another group at risk are pregnant women.

Other clinical manifestations of zinc deficiency than mentioned above are suppressed immunity, poor healing, dermatitis and impairments in neuropsychological functions.

The diagnosis of zinc deficiency is a problem while sensitive indices of zinc status are lacking. The most widely used indices of zinc status are levels of zinc in plasma or serum. These parameters may be decreased in cases of severe and moderate deficiency. Dietary data and indirect measures of bone health indicate that the bioavailability of calcium is important when habitual intakes are low, especially during periods of bone growth or loss. Bioavailability of calcium was found to be low in diets high in unrefined cereals, that are rich in phytate.

Cereals, together with oil seeds and legumes, supply a majority of the dietary protein, calories, vitamins, and minerals to the bulk population. Phosphorous, potassium, magnesium, calcium and traces of iron and other minerals are found in cereals. Barley and wheat provide 50 and 36 mg Ca/100 g respectively. Barley provides 6 mg of iron per 100 g; millet provides 6.8; oats, 4.6 and wheat, 3.1.

Inositol hexaphosphate, IP6, is ubiquitous in nature and comprises the bulk of eukaryotic cell inositol phosphate content. In plants, IP6 constitutes the principal storage form of phosphorous, in particular whole grains of cereals and legumes are rich in IP6. In cereals phytate is located in bran and germ, whereas in legume seeds phytate occurs in the protein bodies in the endosperm. The highly negatively charged IP6 forms various complexes with minerals and proteins, commonly known as phytate. Phytate is considered an anti-nutrient due to the formation of precipitated complexes that strongly reduces the absorption of essential dietary minerals such as iron, zinc, calcium and magnesium. A dose dependent inhibition of iron, zinc and calcium absorption by phytate has been demonstrated in humans. Moreover, inositol penta phosphate has been identified as an inhibitor of iron and zinc absorption. In addition, it has been suggested that IP6 may influence negatively on solubility, digestibility and activity of proteins such as digesting enzymes Reddy et al. Reduction in antinutritional and toxic components in plantfoods by fermentation. Food Res. Int. 27, 281-290. Degradation of phytate to low levels in cereal and legume meals was demonstrated to markedly improve iron and zinc absorption in humans. To improve iron absorption the degradation has to be virtually complete. Thus, once phytate is degraded these foods become a good source of dietary minerals.

Degradation of phytate is possible with phytases. Native phytase in some cereals may be active during traditional processing such as soaking, germination, malting and fermentation and phytate degradation can be improved by process optimization, selection of specific starter cultures, or phytase can be added. It has also been demonstrated that phytate degradation in the stomach and small intestine of humans occurs as a result of activity of dietary phytase of plant or microbial origin thereby improving iron and zinc absorption. Consumption of foods containing active phytase enzymes is therefor an alternative to phytate removal during food processing. The plant phytase is less stable than the microbial phytase at the physiological conditions of the gastro-intestine and is therefor less effective in this respect.

Since IP6 is such a common compound in nature some microorganisms would be expected to have the ability to degrade IP6 and utilize the hydrolyzed phosphorous. This is true for certain bacteria, Riedereret al. (1991) Removal of N-glycosylation sites of the yeast acid phosphatase severely affects protein folding. J. Bacteriol. 173, 3539-3546 and many fungi, Shieh et al. (1968) Survey of microorganism for the production of extracellular phytase. Appl. Microbiol. 16, 1348-1351; Dvoráková et al. (1997) Characterization of phytase produced by Aspergillus niger. Folia Microbiol. 42, 349-352; Wyss et al. (1998) Comparison of the thermostability properties of three acid phosphatases from molds: Aspergillus fumigatus phytase, A. niger phytase, and A. niger pH 2.5 acid phosphatase. Appl. Environ. Microbiol. 64, 4446-4451, that are well known to synthesize secretory so called phytases, i.e. phosphatases hydrolyzing IP6 to inositol pentaphosphate (IP5) and inorganic ortho-phosphate (Pᵢ). Phytases, derived from Aspergillus sp are frequently used in animal feeds to improve phosphorous and mineral availability, and much research is devoted to further improve these by e.g. molecular enzyme engineering, Wyss et al. (1999) Biophysical characterization offungal phytases (myo-inositol hexakisphosphate phosphohydrolases): molecular size, glycosylation pattern, and engineering ofproteolytic resistance. Appl. Environ. Microbiol. 65, 359-366. For humans, however, no corresponding approved (food grade) enzyme is available. One way to improve the mineral state in vulnerable human populations may be to explore yeasts, that with their GRAS- (generally regarded as safe) status are preferable microorganisms in human foods. The natural yeast phytase activity during for instance bread leavening, however, seems too low to significantly influence the iron absorption when eating foods fermented by yeast, such as bread, Tüirk et al. (1996) Reduction in the levels ofphytate during wholemeal bread making; Effect of yeast and wheat phytases. J. Cereal Science. 23, 257-264.

It is further noted that it is known from the patent literature that yeasts, such as Pichia rhodanensis (JP2000050864), Arxula adeninivorans (JP2000050863), Candida boidinii (EP 0 931 837), Saccharomyces cerevisiae (WO2001036607) may comprise genes for expression of phytase.

Greiner et al, J. Agric. Food Chem. (2001) 49(5), 2228-2233 relates to production of stereospecific myo-inositol hexaphosphate by using baker's yeast phytase.

Nakamura, et al, Biosci. Biotechnol. Biochem (2000), 64(4), 841-844 describe dephosphorylation of inositol using baker's yeast.

Turk et al, J. Agric. Food Chem. (2000), 48(1), 100-104 describes that phosphorous is stored in plants as phytates, and discusses the negative effect on mineral bioavailability and shows the ability of Saccharomyces cerevisiae to reduce phytate by its phytase production.

The PHO gene family has been extensively studied in Saccharomyces cerevisiae, Yoshida et al. (1989) Function of the PHO regulatory genes for repressible acid phosphatase synthesis in Saccharomyces cerevisiae. Mol. Gen. Genet. 217, 40-46; Ogawa et al. (2000) New components of a system for phosphate accumulation and polyphosphate metabolism in Saccharomyces cerevisiae revealed by genomic expression analysis. Mol. Biol. Cell. 11, 4309-4321, however, not in the context of IP6 as the substrate. The secretory acid phosphatases encoded by the structural genes PH03, PH05, PH010 and PH011 all seem to be aimed for hydrolyzing extracellular organic phosphorous compounds allowing the yeast to grow in the absence of inorganic phosphate. All except PH03 are repressed by inorganic phosphate, whereas PH03 is repressed by thiamine present in the external environment, Praekelt et al. (1994) Regulation of TH14 (MOL1), a thiamine-biosynthetic gene of Saccharomyces cerevisiae. Yeast. 10, 481-490. PH03 is therefore suggested to primarily hydrolyze phosphate from thiamine phosphate or thiamine pyro-phosphate; Nosakaet al. (1989) A possible role for acid phosphatase with thiamin-binding activity encoded by PHO3 in yeast. FEMS Microbiol. Lett. 51, 55-59; Nosaka (1990) High affinity of acid phosphatase encoded by PHO3 gene in Saccharomyces cerevisiae for thiamin phosphates. Biochim. Biophys. Acta. 1037, 147-154

PH05 is often described as responsible for the major fraction of secretory acid phosphatase activity whereas PH010 and PH011 encode a minor fraction of secretory phosphatase, Rogers, D. T., Lemire, J. M. and Bostian, K. A. (1982) Acid phosphatase polypeptides in Saccharomyces cerevisiae are encoded by a differentially regulated multigene family. Proceedings of the National Academy of Sciences of the United States of America. 79, 2157-2161; Lemire et al. (1985) Regulation of repressible acid phosphatase gene transcription in Saccharomyces cerevisiae. Mol. Cell Biol. 5, 2131-2141, that may be lowly and constitutively expressed. In addition to the enzymes, several components within the PHO system are regulatory proteins, such as Pho4p and Pho2p which are transcriptional activators for PH05, PH010 and PH011, Vogel et al. (1989) The two positively acting regulatory proteins Pho2p and Pho4p physically interact with PHO5 upstream activation regions. Mol. Cell. Biol. 9, 2050-2057

A single study approaches the question of level of phytase activity derived from specific S. cerevisiae phosphatase encoding genes, Moore et al. (1995) Molecular cloning, expression and evaluation of phosphohydrolases for phytate-degrading activity. J. Ind. Microbiol. 14, 396-402. In that work, however, the yeast genes were all expressed in Aspergillus leading to uncertainty in factors such as copy number, levels of glycosylation and secretion as well as to what extent the genes contribute to phytase activity in S. cerevisiae. The opposite approach was taken by Han et al, Han et al. (1999) Expression of an Aspergillus niger phytase gene (phyA) in Saccharomyces cerevisiae. Appl. Environ. Microbiol. 65, 1915-1918, who instead expressed the Aspergillus niger phytase gene (phyA) in S. cerevisiae, to explore an alternative expression system for a well-known phytase.

### SUMMARY OF INVENTION

Broadly, the invention includes a process for preparing a Saccharomyces cerevisiae yeast strain having improved phytase activity comprising modifying the strain by inserting one or more of the PHO5, PHO10, PHO11, DIA3, PHO2 and/or PHO4 genes, and/or by deleting one or more of the negative regulatory genes PHO80, PHO85, and/or PHO23, whereby the insertion is either chromosomal or by transforming the strain using a plasmid containing the gene/s. The modified strain overxpresses one or more the PHO5, PHO10, PHO11, DIA3, PHO2 and/or PHO4 genes.

In a preferred embodiment the modified Saccharomyces cerevisiae overexpresses PHO5.

In another preferred embodiment the modified Saccharomyces cerevisiae overexpresses PHO10.

In a further preferred embodiment the modified Saccharomyces cerevisiae overexpresses PHO11.

In another preferred embodiment the modified Saccharomyces cerevisiae overexpresses PHO2.

In another preferred embodiment the modified Saccharomyces cerevisiae overexpresses PHO4.

In another preferred embodiment the modified Saccharomyces cerevisiae overexpresses DIA3.

In a further preferred embodiment of the invention the modified Saccharomyces cerevisiae is a strain, wherein the negatively regulating gene PHO80 has been deleted to form a mutant pho80Δ.

In order to further increase phytase activity, other genes belonging to the PHO family like the genes encoding for phytase or some transcription regulators, such as PHO2 and PHO4, may eventually have to be over-expressed alone or in combination with other genetic modifications, such as pho80 or pho85 mutations.

In another preferred embodiment of the invention the modified Saccharomyces cerevisiae is a strain, wherein the negatively regulating gene PHO85 has been deleted to form a mutant pho85Δ.

In a further preferred embodiment of the invention the modified Saccharomyces cerevisiae over expresses the positively regulating gene PHO2.

In a further preferred embodiment of the invention the modified Saccharomyces cerevisiae over expresses the positively regulating gene PHO4.

In accordance with a further aspect of the invention, the invention includes a fermented product having been fermented using a Saccharomyces cerevisiae strain identified above, and having a reduced content of inositol hexaphosphate.

In one embodiment the fermented product is a bakery product.

In accordance with another embodiment the fermented product is a gruel product, preferably for infants.

In a further embodiment the fermented product is a brewed product.

In yet another aspect of the invention, a method for the preparation of phytase is provided wherein one or more of the strains identified above are grown in a growth medium that facilitates phytase expression, whereupon produced phytase is isolated.

In accordance with a further aspect of the invention such a phytase preparation can be used as an additive in foodstuffs or feedstuffs to improve the utilization of minerals.

In accordance with a further aspect of the invention such a phytase preparation can be used to produce specific isomers of lower inositol phosphates

In yet another aspect of the invention, the invention provides a method for the preparation of phytase wherein one or more of the strains identified above are grown in a medium under conditions optimized for phytase expression whereupon lower inositol phosphates are produced as a result of yeast phytase activity on added inositol hexaphosphate.

Besides fermented products for human use the present Saccharomyces cerevisiae strains can be used for the production of animal feedstuffs to increase mineral uptake, and to reduce the phosphate amounts in faeces. Phosphorous in the form of IP6 can not be resorbed in the intestines and thus animal feedstuffs are enriched using inorganic phosphates. IP6 together with excess of phosphates follow the faeces, which leads to high amounts/concentrations of phosphorous in the manure.

Besides the foodstuff and feedstuff applications the enzyme phytase can be used for the manufacture of specific inositol phosphates for pharmacological use. One example is 1,2,6IP3, which may stimulate the release of insuline, to reduce thrombocyte aggregation and to have anti-inflammatory properties. Such inositol phosphates may be developed into pharmaceuticals.

The enzyme phytase can also be added to foodstuffs and feedstuffs to obtain the effects specified above. Thus a further aspect of the present invention is to grow the modified Saccharomyces cerevisiae and to isolate the phytase enzyme produced. Such isolation is readily carried out as the phytase is present extracellularly. Primarily the addition of phytase is made to improve availability of iron and zinc to the body.

The invention also includes a modified strain of Saccharomyces cereviaisae exhibiting increased phytase activity, the strains being designated as YM-p5 and YD80.

### BRIEF DESCRIPTION OF THE DRAWINGS

FIG. 1 is a graph depicting the growth of S. cerevisiae SKQ2n (squares) and extracellular concentration of inositol hexaphosphate (IP6; triangles), measured by HPIC, as a function of time;

FIG. 2 is a graph depicting the chromatographic profiles (HPIC) of chemical acid hydrolysate of sodium phytate (first line), and supernatant from S. cerevisiae cultures (second and third lines) after 15 h of growth;

FIGS. 3A and 3B are graphs depicting the growth (squares) of S. cerevisiae parent strain YS18 (open symbols) and S. cerevisiae YMR4 pho5Δpho3Δ double deletion mutant (filled symbols) and extracellular concentration of IP6 (circles) as determined by HPIC;

FIG. 4 is a graph depicting the concentration of inositol hexaphosphate (IP6) in yeast culture supernatant as a function of growth in CBS medium with IP6 as the sole phosphorous source;

FIG. 5 is a graph depicting the concentration of inositol hexaphosphate (IP6) in yeast culture supernatant as a function of growth in complex YPD medium with IP6 as the added phosphorous source;

FIG. 6 is a vector construct consisting of plasmid pYX212 containing the insert PHO5 used for transformation of YS18 and YMR4;

FIGS. 7A and 7B are graphs depicting the extracellular concentration of IP6 as a function of growth time of S. cerevisiae YMR4 (A) and YS18 (B), with and without the PHO5 containing plasmid pYX212;

FIG. 8 is a graph depicting a direct comparison of phytate degrading capacity between PHO5 overexpressing strains, and pho80Δ and pho85Δ deletions mutants;

FIG. 9 is a graph depicting phytate degrading capacity of pho2Δ deletion mutant;

FIG. 10 is a graph depicting the extracellular concentration of para-nitrophenyl phosphate (pNPP, squares) and IP6 (triangles) as a function of growth of S. cerevisiae SKQ2n in CBS medium with 2% glucose as carbon and energy source;

FIG. 11. is a graph depicting the content of inositol hexaphosphate (IP6, triangles) and inorganic phosphate (Pᵢ; squares), expressed as μmol per gram wet dough, as a function of leavening time in two types of wheat based dough; and

FIG. 12 is a graph depicting phytate degradation capacity of the pho80Δ deletion mutant SD80 (YS18 without PHO80) compared with S. cerevisiae YS18 in YPD supplemented with 0.25 mM IP6 and 30 mM Pi.

### DETAILED DESCRIPTION OF INVENTION

The invention will now be described in reference to the following non-limiting examples.

Materials and Methods

Organisms

Saccharomyces cerevisiae SKQ2n, Bataille et al. (1987) Identification of polypeptides of the carbon metabolism machinery on the two-dimensional map of Saccharomyces cerevisiae. Location of 23 additional polypeptides. Yeast. 3, 11-21,. S. cerevisiae YS18 (MATα; his3-11, 3-15; leu2-3, 2-112; ura3Δ5; canR ) and S. cerevisiae YMR4 were used in most experiments as indicated. YS18 and YMR4 were previously constructed by Riederer and Hinnen; Riederer et al. (1991) Removal of N-glycosylation sites of the yeast acid phosphatase severely affects protein folding. J. Bacteriol. 173, 3539-3546. YS18 and YMR4 were in the present work both transformed with a PHO5 containing plasmid (see below) and the resulting transformant strains were designated YS-p5 and YM-p5, respectively. Strain YS-p5 was deposited with DSMZ—Deutsche Sammilung von Mikroorganismen und Zellkulturen GmbH, Mascheroder Weg 1b, 38124 Braunschweig, Germany on Apr. 18, 2002 and was assigned accession number DSM-No. 14929.

YS18 and YMR4 were further deleted for PHO10 yielding strains named YS10 and YM10, respectively. YS18 was also used for construction of the pho80Δ strain labeled YD80. In addition, pho80Δ, pho85Δ and pho2Δ mutant strains were retrieved from the German EUROSCARF collection. pho80 (YOL001w), pho85 (YPL031c) and pho2 (YDL106c) strains originates from the laboratory BY4741 strain (MATa; his3Δ1, leu2Δ0, met15Δ0, ura3Δ0). The PHO80, PHO85 and PHO2 genes were deleted with kan::MX4 disruption cassette. Yeast cultures were maintained on YPD-plates (yeast extract 10 g, peptone 20 g, glucose 20 g and agar 20 g in one liter of water) and stored long-term in glycerol at −70° C. Escherichia coli DH5α was used for propagating the plasmid containing the inserted PHO5 (see below). The yeast strains and their expression profiles of relevant genes are listed in Table 1.

TABLE 1
Strains of Saccharomyces cerevisiae used in this study, their relevant genotype and expression
profiles of secretory phosphatases in repressing and non-repressing media.

| | | Secretory phosphates | ^{a}Secretory phosphates | |
|---|---|---|---|---|
| | Relevant | expressed in IP₆ | expressed in IP₆ + P₁ | Reference for |
| Strain | genotype | medium | medium | strain |
| SKQ2n | all PHO genes | Pho5p, Pho10p, Pho11p | none | Bataille et al. |
| | intact | | | (1987) |
| YS18 | all PHO genes | Pho5p, Pho10p, Pho11p | none | Riederer et et al. |
| | intact. Parent | | | (1991) |
| | strain for YMR4 | | | |
| YMR4 | pho5,3::uraΔ1 | Pho10p, Pho11p | none | Riedereret et al. |
| | | | | (1991) |
| YM-p5 | pho5,3::uraΔ1 | Pho5p^{b}, Pho10p, | Pho5p^{b} | This work |
| | pYX-PHO5 | Pho11p | | |
| YS-p5 | pYX-PHO5 | Pho5p^{b}, Pho5p, Pho10p, | Pho5p^{b} | This work |
| | | Pho11p | | |
| YM10 | pho5,3::ura3Δ1 | Pho11p | none | This work |
| | pho10::his3Δ | | | |
| YS10 | pho10::his3Δ | Pho5p, Pho11p | none | This work |
| BY4741 | All PHO genes | Pho5p, Pho10p, Pho11p | none | EUROSCARF |
| | intact | | | |
| YOL001w | pho80Δ | Pho5p, Pho10p, Pho11p | Pho5p, Pho10p, Pho11p | EUROSCARF |
| YPL031c | pho85Δ | Pho5p, Pho10p, Pho11p | Pho5p, Pho10p, Pho11p | EUROSCARF |
| YDL106c | pho2Δ | none | none | EUROSCARF |
| YD80 | pho80Δ::hph | Pho5p, Pho10p, Pho11p | Pho5p, Pho10p, Pho11p | This work |

^{a}A small constitutive expression may occur
^{b}On the plasmid pYX212 under the control of TP11 promoter

Growth Media and Culture Conditions

The growth medium used in most experiments was a modified version of the defined yeast minimal medium developed by CBS (Centralbueau voor Schimmelcultures, Delft, the Netherlands) which has been described previously, Albers et al. (1996) Influence of the nitrogen source on Saccharomyces cerevisiae anaerobic growth and product formation. Appl. Environ. Microbiol. 62, 3187-3195 using ammonium sulfate (7.5 mg/ml) as nitrogen source. As phosphorus source, either IP6 in the form of sodium phytate (C₆H₆(OPO₃Na₂)₆: 0.5 or 0.25 mg/ml), inorganic phosphate (KH₂PO₄: 3.5 mg/ml), para-nitrophenyl phosphate (PNPP; to a final concentration of 2.25 or 0.25 mM), or combinations of these were used. Depending on the phosphorous source used the media were designated CBSIP6, CBSPᵢ, CBSIP6+Pᵢ and CBSpNPP. CBSIP6 and CBSpNPP were supplemented with KCl (3 mg/ml) to compensate for the potassium present in the inorganic P-source (KH₂PO₄), but absent in the organic P-sources. In all experiments glucose (either 2% (w/v) or 1% (w/v) as indicated) was used as carbon and energy source. When appropriate, histidine, leucine and uracil were supplied at 120 mg/l. Most experiments were also performed in YPD media (per liter: yeast extract, 10 g; peptone 20 g and glucose 20 g) supplemented with the appropriate P-sources as indicated in results and figure legends. Succinic acid/NaOH, pH 5.3, was used as buffer in the CBS based media. Medium components were autoclaved, with the exception for IP6, pNPP, vitamins, growth factors and FeCl₂, which were sterilized by filtration through a 0.2 μm filter. Experimental cultures containing 100 ml in 250 ml E-flasks were inoculated with primary cultures grown for approximately 20h in medium of the same composition as the respective experiment culture. The inoculation level was set to OD₆₁₀=0.2. The experimental cultures were grown at 30° C. in a rotary shaker set to 210 rpm. The growth was monitored as optical density at 610 nm (OD₆₁₀) using a spectrophotometer (Hitachi, model U-100). Experiments aimed at PHO3 expression were performed in CBS medium as described above with the exception of excluding thiamine (to avoid repression of the thiamine phosphatase PHO3), with and without addition of thiamine monophosphate.

Construction of PHO5 Overexpressing Strain.

Chemicals and enzymes for the procedures were purchased from New England Biolabs Inc., USA, and the PCR primers used for amplification of PHO5 were purchased from Life Technologies AB, Täby, Sweden. Extraction of genomic DNA from S. cerevisiae YS18 was carried out according to standard procedures. The nucleotide sequences of the primers are shown in upper case letters and the added restriction enzyme sites are shown in lower case letters.

Primers for Overexpression of PH05

Forward primer:
5′:CGGAATTCATGTTTAAATCTGTTGTTTATTC,
appended as SEQ ID NO: 1
Backwards primer:
3′:CGCTCGAGCTATTGTCTCAATAGACTGGC,
appended as SEQ ID NO: 2

Underlined sequences are EcoRI and Aval (yields end compatible to XhoI used to cut the pYX212 plasmid) restriction sites, respectively. The remaining sequences complement the beginning (from ATG) and end of PHO5.

The PCR reaction was performed in 100 μl reaction mixtures, using VENT polymerase and was carried out according to standard procedures.

The PCR produced PHO5 was purified with a PCR purification kit (Qiagen, Cat, No. 28104, Merck Eurolab AB, Sweden) according to the manufacturer's protocol and 50 μl was cut with AvaI and EcoRI in EcoRI buffer for 12 h at 37° C. The cut PCR product was loaded on a preparative agarose gel and run together with a DNA ladder in TAE buffer. The appropriate band was cut out and purified using DNA gel extraction kit (Qiagen, Cat. No. 28704). A 20 μl ligation mixture was prepared by mixing 16 μl purified insert (PHO5), 2 μl T4 DNA ligase buffer (10×) 1 μl ligase and 2 μl vector DNA (pYX212). The plasmid pYX212 containing the selection markers Amp^{R} for E. coli and URA3 for S. cerevisiae had previously been cut with EcoRI and XhoI (yields ends compatible with AvaI). Two μl of the DNA construct was added to 40 μl of cold competent E. coli DH5α. and transformed by electroporation using a Gene Pulser II (Biorad) set to the 25 μF capacitator, 2.5 kV and the pulse controller to 200 Ω. Cells were plated on NB plates containing 100 μg/ml ampicillin. Clones were cultivated over night in liquid NB medium and plasmids were prepared according to standard procedures.

Sequence of the Plasmid pYX212 with Insert PHO5:

GAATTCATGTTTAAATCTGTTGTTTATTCAATTTTAGCCGCTTCTTTGGC
CAATGCAGGTACCATTCCCTTAGGCAAACTAGCCGATGTCGACAAGATTG
GTACCCAAAAAGATATCTTCCCATTTTTGGGTGGTGCCGGACCATACTAC
TCTTTCCCTGGCGACTATGGTATTTCTCGTGATTTGCCTGAAGGTTGTGA
AATGAAGCAACTGCAAATGGTTGGTAGACATGGTGAAAGATACCCTACTG
TCAGTCTGGCTAAGACTATCAAGAGTACATGGTATAAGTTGAGCAATTAC
ACTCGTCAATTCAACGGCTCATTGTCATTCTTGAACGATGATTACGAGTT
TTTCATCCGTGATGACGATGATTTGGAAATGGAAACCACTTTTGCCAACT
CGGACGATGTTTTGAACCCATACACTGGTGAAATGAACGCCAAGAGACAT
GCTCGTGACTTCTTGGCTCAATACGGTTACATGGTCGAAAACCAAACCAG
TTTCGCCGTTTTTACCTCTAATTCTAAGAGATGTCATGACACTGCTCAAT
ATTTCATTGATGGTTTAGGTGACCAATTCAACATCACCTTGCAGACTGTC
AGTGAAGCTGAATCCGCTGGTGCCAACACTTTGAGTGCTTGTAACTCATG
TCCTGCTTGGGACTACGATGCCAATGATGACATTGTAAATGAATACGACA
CAACCTACTTGGATGACATTGCCAAGAGATTGAACAAGGAAAACAAGGGT
TTGAACTTGACCTCAACTGACGCTAGTACTTTATTCTCGTGGTGTGCATT
TGAAGTGAACGCTAAAGGTTACAGTGATGTCTGTGATATTTTCACCAAGG
ATGAATTAGTCCATTACTCCTACTACCAAGACTTGCACACTTATTACCAT
GAGGGTCCAGGTTACGACATTATCAAGTCTGTCGGTTCCAACTTGTTCAA
TGCCTCAGTCAAATTATTAAAGCAAAGTGAGATTCAAGACCAAAAGGTTT
GGTTGAGTTTTACCCACGATACCGATATCCTAAACTTTTTGACCACCGCT
GGTATAATTGACGACAAAAACAACTTAACTGCCGAATACGTTCCATTCAT
GGGCAACACTTTCCACAGATCCTGGTACGTTCCTCAAGGTGCTCGTGTCT
ACACCGAAAAATTCCAATGTTCTAACGACACCTACGTCAGATACGTCATT
AACGATGCTGTTGTTCCAATTGAAACCTGTTCCACTGGTCCAGGGTTCTC
TTGTGAAATCAATGACTTCTACGACTATGCTGAAAAGAGAGTAGCCGGTA
CTGACTTCCTAAAGGTCTGTAACGTCAGCAGCGTCAGTAACTCTACTGAA
TTGACCTTCTACTGGGACTGGAACACTACTCATTACAACGCCAGTCTATT
GAGACAATAGCCCGGGTATCCGTATGATGTGCCTGACTACGCATGATATC
TCGAGCTCAGCTAGCTAACTGAATAAGGAACAATGAACGTTTTTCCTTTC
TCTTGTTCCTAGTATTAATGACTGACCGATACATCCCTTTTTTTTTTTGT
CTTTGTCTAGCTCCAGCTTTTGTTCCCTTTAGTGAGGGTTAATTCAATTC
ACTGGCCGTCGTTTTACAACGTCGTGACTGGGAAAACCCTGGCGTTACCC
AACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGG
CGAATGGCGCGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGGTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCCTGATGCGGTATTTTCTCCTTACGCATCTGTG
CGGTATTTCACACCGCATAGGGTAATAACTGATATAATTAAATTGAAGCT
CTAATTTGTGAGTTTAGTATACATGCATTTACTTATAATACAGTTTTTTA
GTTTTGCTGGCCGCATCTTCTCAAATATGCTTCCCAGCCTGCTTTTCTGT
AACGTTCACCCTGTACCTTAGCATCCCTTCCCTTTGCAAATAGTCCTCTT
CCAACAATAATAATGTCAGATCCTGTAGAGACCACATCATCCACGGTTCT
ATACTGTTGACCCAATGCGTCTCCCTTGTCATCTAAACCCACACCGGGTG
TCATAATCAACCAATCGTAACCTTCATCTCTTCCACCCATGTCTCTTTGA
GCAATAAAGCCGATAACAAAATCTTTGTCGCTCTTCGCAATGTCAACAGT
ACCCTTAGTATATTCTCCAGTAGATAGGGAGCCCTTGCATGACAATTCTG
CTAACATCAAAAGGCCTCTAGGTTCCTTTGTTACTTCTTCTGCCGCCTGC
TTCAAACCGCTAACAATACCTGGCCCCAGCACACCGTGTGCATTCGTAAT
GTCTGCCCATTCTGCTATTCTGTATACACCCGCAGAGTACTGCAATTTGA
CTGTATTACCAATGTCAGCAAATTTTCTGTCTTCGAAGAGTAAAAAATTG
TACTTGGCGGATAATGCCTTTAGCGGCTTAACTGTGCCCTCCATCGAAAA
ATCAGTCAATATATCCACATGTGTTTTTAGTAAACAAATTTTGGGACCTA
ATGCTTCAACTAACTCCAGTAATTCCTTGGTGGTACGAACATCCAATGAA
GCACACAAGTTTGTTTGCTTTTCGTGCATGATATTAAATAGCTTGGCAGC
AACAGGACTAGGATGAGTAGCAGCACGTTCCTTATATGTAGCTTTCGACA
TGATTTATCTTCGTTTCCTGCAGGTTTTTGTTCTGTGCAGTTGGGTTAAG
AATACTGGGCAATTTCATGTTTCTTCAACACTACATATGCGTATATATAC
CAATCTAAGTCTGTGCTCCTTCCTTCGTTCTTCCTTCTGTTCGGAGATTA
CCGAATCAAAAAAATTTCAAAGAAACCGAAATCAAAAAAAAGAATAAAAA
AAAAATGATGAATTGAATTGAAAAGCTGTGGTATGGTGCACTCTCAGTAC
AATCTGCTCTGATGCCGCATAGTTAAGCCAGCCCCGACACCCGCCAACAC
CCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTC
ATCACCGAAACGCGCGAGACGAAAGGGCCTCGTGATACGCCTATTTTTAT
AGGTTAATGTCATGATAATAATGGTTTCTTAGACGTGCGGCCGCTCTAGA
ACTAGTGGATCAATTCCACGGACTATAGACTATACTAGTATACTCCGTCT
ACTGTACGATACACTTCCGCTCAGGTCCTTGTCCTTTAACGAGGCCTTAC
CACTCTTTTGTTACTCTATTGATCCAGCTCAGCAAAGGCAGTGTGATCTA
AGATTCTATCTTCGCGATGTAGTAAAACTAGCTAGACCGAGAAAGAGACT
AGAAATGCAAAAGGCACTTCTACAATGGCTGCCATCATTATTATCCGATG
TGACGCTGCAGCTTCTCAATGATATTCGAATACGCTTTGAGGAGATACAG
CCTAATATCCGACAAACTGTTTTACAGATTTACGATCGTACTTGTTACCC
ATCATTGAATTTTGAACATCCGAACCTGGGAGTTTTCCCTGAAACAGATA
GTATATTTGAACCTGTATAATAATATATAGTCTAGCGCTTTACGGAAGAC
AATGTATGTATTTCGGTTCCTGGAGAAACTATTGCATCTATTGCATAGGT
AATCTTGCACGTCGCATCCCCGGTTCATTTTCTGCGTTTCCATCTTGCAC
TTCAATAGCATATCTTTGTTAACGAAGCATCTGTGCTTCATTTTGTAGAA
CAAAAATGCAACGCGAGAGCGCTAATTTTTCAAACAAAGAATCTGAGCTG
CATTTTTACAGAACAGAAATGCAACGCGAAAGCGCTATTTTACCAACGAA
GAATCTGTGCTTCATTTTTGTAAAACAAAAATGCAACGCGAGAGCGCTAA
TTTTTCAAACAAAGAATCTGAGCTGCATTTTTACAGAACAGAAATGCAAC
GCGAGAGCGCTATTTTACCAACAAAGAATCTATACTTCTTTTTTGTTCTA
CAAAAATGCATCCCGAGAGCGCTATTTTTCTAACAAAGCATCTTAGATTA
CTTTTTTTCTCCTTTGTGCGCTCTATAATGCAGTCTCTTGATAACTTTTT
GCACTGTAGGTCCGTTAAGGTTAGAAGAAGGCTACTTTGGTGTCTATTTT
CTCTTCCATAAAAAAAGCCTGACTCCACTTCCCGCGTTTACTGATTACTA
GCGAAGCTGCGGGTGCATTTTTTCAAGATAAAGGCATCCCCGATTATATT
CTATACCGATGTGGATTGCGCATACTTTGTGAACAGAAAGTGATAGCGTT
GATGATTCTTCATTGGTCAGAAAATTATGAACGGTTTCTTCTATTTTGTC
TCTATATACTACGTATAGGAAATGTTTACATTTTCGTATTGTTTTCGATT
CACTCTATGAATAGTTCTTACTACAATTTTTTTGTCTAAAGAGTAATACT
AGAGATAAACATAAAAAATGTAGAGGTCGAGTTTAGATGCAAGTTCAAGG
AGCGAAAGGTGGATGGGTAGGTTATATAGGGATATAGCACAGAGATATAT
AGCAAAGAGATACTTTTGAGCAATGTTTGTGGAAGCGGTATTCGCAATAT
TTTAGTAGCTCGTTACAGTCCGGTGCGTTTTTGGTTTTTTGAAAGTGCGT
CTTCAGAGCGCTTTTGGTTTTCAAAAGCGCTCTGAAGTTCCTATACTTTC
TAGAGAATAGGAACTTCGGAATAGGAACTTCAAAGCGTTTCCGAAAACGA
GCGCTTCCGAAAATGCAACGCGAGCTGCGCACATACAGCTCACTGTTCAC
GTCGCACCTATATCTGCGTGTTGCCTGTATATATATATACATGAGAAGAA
CGGCATAGTGCGTGTTTATGCTTAAATGCGTACTTATATGCGTCTATTTA
TGTAGGATGAAAGGTAGTCTAGTACCTCCTGTGATATTATCCCATTCCAT
GCGGGGTATCGTATGCTTCCTTCAGCACTACCCTTTAGCTGTTCTATATG
CTGCCACTCCTCAATTGGATTAGTCTCATCCTTCAATGCTATCATTTCCT
TTGATATTGGATCATATGCATAGTACCGAGAAACTAGTGCGAAGTAGTGA
TCAGGTATTGCTGTTATCTGATGAGTATACGTTGTCCTGGCCACGGCAGA
AGCACGCTTATCGCTCCAATTTCCCACAACATTAGTCAACTCCGTTAGGC
CCTTCATTGAAAGAAATGAGGTCATCAAATGTCTTCCAATGTGAGATTTT
GGGCCATTTTTTATAGCAAAGATTGAATAAGGCGCATTTTTCTTCAAAGC
TGCGGCCGCACTCTCACTAGTACGTCAGGTGGCACTTTTCGGGGAAATGT
GCGCGGAACCCCTATTTGTTTATTTTTCTAAATACATTCAAATATGTATC
CGCTCATGAGACAATAACCGTGATAAATGCTTCAATAATATTGAAAAAGG
AAGAGTATGAGTATTCAACATTTCCGTGTCGCCCTTATTCCCTTTTTTGC
GGCATTTTGCCTTCCTGTTTTTGCTCACCCAGAAACGCTGGTGAAAGTAA
AAGATGCTGAAGATCAGTTGGGTGCACGAGTGGGTTACATCGAACTGGAT
CTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTA
TTGACGCCGGGCAAGAGCAACTCGCTCGCCGCATACACTATTCTCAGAAT
GACTTGGTTGAGTACTCACCAGTCACAGAAAAGCATCTTACGGATGGCAT
GACAGTAAGAGAATTATGCAGTGCTGCCATAACCATGAGTGATAACACTG
CGGCCAACTTACTTCTGACAACGATCGGAGGACCGAAGGAGCTAACCGCT
TTTTTGGACAACATGGGGGATCATGTAACTCGCCTTGATCGTTGGGAACC
GGAGCTGAATGAAGCCATACCAAACGACGAGCGTGACACCACGATGCCTG
TAGCAATGGCAACAACGTTGCGCAAACTATTAACTGGCGAACTACTTACT
CTAGCTTCCCGGCAACAATTAATAGACTGGATGGAGGCGGATAAAGTTGC
AGGACCACTTCTGCGCTCGGCCCTTCCGGCTGGCTGGTTTATTGCTGATA
AATCTGGAGCCGGTGAGCGTGGGTCTCGCGGTATCATTGCAGCACTGGGG
CCAGATGGTAAGCCCTCCCGTATCGTAGTTATCTACACGACGGGGAGTCA
GGCAACTATGGATGAACGAAATAGACAGATCGCTGAGATAGGTGCCTCAC
TGATTAAGCATTGGTAACTGTCAGACCAAGTTTACTCATATATACTTTAG
ATTGATTTAAAACTTCATTTTTAATTTAAAAGGATCTAGGTGAAGATCCT
TTTTGATAATCTCATGACCAAAATCCCTTAACGTGAGTTTTCGTTCCACT
GAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCG
TAGTTAGGCCACCACTTCAAGAACTCTGTAGCACCGCCTACATACCTCGC
TCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCG
GGCTGAACGGGGGGTTCGTGCACACAGCCCAGCTTGGAGCGAACGACCTA
CACCGAACTGAGATACCTACAGCGTGAGCTATGAGAAAGCGCCACGCTTC
CCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTA
CGGTTCCTGGCCTTTTGCTGGCCTTTTGCTCACATGTTCTTTCCTGCGTT
ATCCCCTGATTCTGTGGATAACCGTATTACCGCCTTTGAGTGAGCTGATA
CCGGTCGCCGCAGCCGAACGACCGAGCGCAGCGAGTCAGTGAGCGAGGAA
GCGGAAGAGCGCCCAATACGCAAACCGCCTCTCCCCGCGCGTTGGCCGAT
TCATTAATGCAGCTGGCACGACAGGTTTCCCGACTGGAAAGCGGGCAGTG
AGCGCAACGCAATTAATGTGAGTTACCTCACTCATTAGGCACCCCAGGCT
TTACACTTTATGCTTCCGGCTCCTATGTTGTGTGGAATTGTGAGCGGATA
ACAATTTCACACAGGAAACAGCTATGACCATGATTACGCCAAGCTCGAAA
TACGACTCACTATAGGGCGAATTGGGTACCGGGCCGGCCGTCGAGCTTGA
TGGCATCGTGGTGTCACGCTCGTCGTTTGGTATGGCTTCATTCAGCTCCG
GTTCCCAACGATCAAGGCGAGTTACATGATCCCCCATGTTGTGAAAAAAA
GCGGTTAGCTCTTCGGTCCTCCGATCGTTGTCAGAAGTAAGTTGGCCGCA
GTGTTATCACTCATGGTTATGGCAGGAACTGCATAATTCTCTTACTGTCA
TGCCATCCGTAAGATGCTTTTCTGTGACTGGTGTACTCAACCAAGTCATT
CTGAGAATAGTGTATGCGGCGACCGAGTTGCTCTTGCCCGGCGTCAACAC
GGGATAATACCGCGCCACATAGCAGAACTTTAAAAGTGCTCATCATTGGA
AAACGTTCTTCGGGGCGAAAACTCTCAAGGATCTTACCGCTGTTGAGATC
CAGTTCGATGTAACCCACTCGTGCACCCAACTGATCTTCAGCATCTTTTA
CTTTCACCAGCGTTTCTGGGTGAGCAAAAACAGGAAGGCAAAATGCCGCA
AAAAAGGGAATAAGGGCGACACGGAAATGTTGAATACTCATACTCTTCCT
TTTTCAATATTATTGAAGCATTTATCAGGGTTATTGTCTCATGAGCGATA
CATATTTGAATGTATTTAGAAAAATAAACAAATAGGGGTTCCGCGCACAT
TTCCCCGAAAAGTGCCACCTGACGTCTAAGAAACCATTATTATCATGACA
TTAACCTATAAAAATAGGCGTATCACGAGGCCCTTTCGTCTTCAAGAATT
GGGGATCTACGTATGGTCATTCTTCTTCAGATTCCCTCATGGAGAAGTGC
GGCAGATGTATATGACAGAGTCGCCAGTTTCCAAGAGACTTTATTCAGGC
ACTTCCATGATAGGCAAGAGAGAAGACCCAGAGATGTTGTTGTCCTAGTT
ACACATGGTATTTATTCCAGAGTATTCCTGATGAAATGGTTTAGATGGAC
ATACGAAGAGTTTGAATCGTTTACCAATGTTCCTAACGGGAGCGTAATGG
TGATGGAACTGGACGAATCCATCAATAGATACGTCCTGAGGACCGTGCTA
CCCAAATGGACTGATTGTGAGGGAGACCTAACTACATAGTGTTTAAAGAT
TACGGATATTTAACTTACTTAGAATAATGCCATTTTTTTGAGTTATAATA
ATCCTACGTTAGTGTGAGCGGGATTTAAACTGTGAGGACCTCAATACATT
CAGACACTTCTGACGGTATCACCCTACTTATTCCCTTCGAGATTATATCT
AGGAACCCATCAGGTTGGTGGAAGATTACCCGTTCTAAGACTTTTCAGCT
TCCTCTATTGATGTTACACTCGGACACCCCTTTTCTGGCATCCAGTTTTT
AATCTTCAGTGGCATGTGAGATTCTCCGAAATTAATTAAAGCAATCACAC
AATTCTCTCGGATACCACCTCGGTTGAAACTGACAGGTGGTTTGTTACGC
ATGCTAATGCAAAGGAGCCTATATACCTTTGGCTCGGCTGCTGTAACAGG
GAATATAAAGGGCAGCATAATTTAGGAGTTTAGTGAACTTGCAACATTTA
CTATTTTCCCTTCTTACGTAAATATTTTTCTTTTTAATTCTAAATCAATC
TTTTTCAATTTTTTGTTTGTATTCTTTTCTTGCTTAAATCTATAACTACA
AAAAACACATACAGG appended as SEQ ID NO: 3.

Underlined sequences show sites for primers
Bolded nucleotides represent the inserted PHO5

The presence of PHO5 was verified by PCR using the same primers as described above as well as by cutting with EcoRV followed by gel electrophoresis, then transformed into yeast strains YS18 and YMR4 using a standard LiOAc S. cerevisiae transformation protocol. Yeasts containing the vector with insert were selected for on uracil negative YNB plates (DIFCO, USA) containing the appropriate amino acids. The resulting transformant strains were named YS-p5 and YM-p5, respectively.

Construction of Deletion Strains

The PHO10 deletions were generated by PCR-mediated gene replacement, Baudin et al. (1993) A simple and efficient for direct gene deletion in Saccharomyces cerevisiae. Nucleic Acids Res. 21, 3329-3330 using HIS5 from Saccharomyces pombe (corresponding to HIS3 in Saccharomyces cerevisiae) as the selectable marker. As template DNA the plasmid pFA6a-HIS3MX6; Wach et al. (1994) New heterologous modules for classical or PCR-based gene disruptions in Saccharomyces cerevisiae. Yeast. 10, 1793-1808 was used.

Primers for Deletion of PHO10

5′:CGATAGATTCAAGCTCAGTTTCGCCTTGGTTGTAAAGTAGGCAGCTG
AAGCTTCGTACGC-3′, appended as SEQ ID NO: 4
5′:GGTCTATTTACTGTTTTAATAAAGTGTCGTTGTAGTGCTTGGGGCAG
ATGATGTCGAGGCG-3′, appended as SEQ ID NO 5

These oligonucleotides were used as primers for construction of deletion cassettes. Underlined sequences are complementary to HIS5 in the template, and non-underlined parts to flanking regions of PHO10. The PHO10 deletions were generated by PCR-mediated gene replacement using HIS5 from Saccharomyces pombe (corresponding to HIS3 in S. cerevisiae) as the selectable marker. As template DNA the plasmid pFA6a-HIS3MX6 was used.

Sequence of the Resulting PCR product used for deletion of PHO10 by homologous recombination:

CGATAGATTCAAGCTCAGTTTCGCCTTGGTTGTAAAGTAGCCAGCTGAAG
CTTCGTACGCTGCAGGTCGACGGATCCCCGGGTTAATTAAGGCGCGCCAG
ATCTGTTTAGCTTGCCTCGTCCCCGCCGGGTCACCCGGCCAGCGACATGG
AGGCCCAGAATACCCTCCTTGACAGTCTTGACGTGCGCAGCTCAGGGGCA
TGATGTGACTGTCGCCCGTACATTTAGCCCATACATCCCCATGTATAATC
ATTTGCATCCATACATTTTGATGGCCGCACGGCGCGAAGCAAAAATTACG
GCTCCTCGCTGCAGACCTGCGAGCAGGGAAACGCTCCCCTCACAGACGCG
TTGAATTGTCCCCACGCCGCGCCCCTGTGAGAAATATAAAAGGTTAGGAT
TTGCCACTGAGGTTCTTCTTTCATATACTTCCTTTTAAATCTTGCTAGGA
TACAGTTCTCACATCACATCCGAACATAAACAACCATGGGTAGGAGGGCT
TTTGTAGAAAGAAATACGAACGAAACGAAAATCAGCGTTGCCATCGCTTT
GGACAAAGCTCCCTTACCTGAAGAGTCGAATTTTATTGATGAACTTATAA
CTTCCAAGCATGCAAACCAAAAGGGAGAACAAGTAATCCAAGTAGACACG
GGAATTGGATTCTTGGATCACATGTATCATGCACTGGCTAAACATGCAGG
CTGGAGCTTACGACTTTACTCAAGAGGTGATTTAATCATCGATGATCATC
ACACTGCAGAAGATACTGCTATTGCACTTGGTATTGCATTCAAGCAGGCT
ATGGGTAACTTTGCCGGCGTTAAAAGATTTGGACATGCTTATTGTCCACT
TGACGAAGCTCTTTCTAGAAGCGTAGTTGACTTGTCGGGACGGCCCTATG
CTGTTATCGATTTGGGATTAAAGCGTGAAAAGGTTGGGGAATTGTCCTGT
GAAATGATCCCTCACTTACTATATTCCTTTTCGGTAGCAGCTGGAATTAC
TTTGCATGTTACCTGCTTATATGGTAGTAATGACCATCATCGTGCTGAAA
GCGCTTTTAAATCTCTGGCTGTTGCCATGCGCGCGGCTACTAGTCTTACT
GGAAGTTCTGAAGTCCCAAGCACGAAGGGAGTGTTGTAAAGAGTACTGAC
AATAAAAAGATTCTTGTTTTCAAGAACTTGTCATTTGTATAGTTTTTTTA
TATTGTAGTTGTTCTATTTTAATCAAATGTTAGCGTGATTTATATTTTTT
TTCGCCTCGACATCATCTGCCCCAAGCACTACAACGACACTTTATTAAAA
CAGTAAATAGACC appended as SEQ ID NO:6

Underlined sequences represent primers (or complementary strand to primer) used Italic shows sequence corresponding to PHO10 (SGD)

The PCR reactions were performed in 100 μl using VENT polymerase, the crude PCR product was purified by cutting the appropriate band from a preparative agorase gel-electrophoresis and YMR4 and YS18 were transformed by the lithium acetate method resulting in strains named YM10 and YS10, respectively. The deletions were verified by PCR on whole yeast colonies as well as on extracted DNA.

Deletion of PHO80 was performed by PCR-based gene disruption, mainly according to Baudin et al., (1993) “A simple and efficient for direct gene deletion in Saccharomyces cerevisiaw.” Nucleic Acids Res. 21, 3329-3330, and Goldstein and McCusker, (1999) “New heterologous modules for classical or PCR-based gene disruptions in Saccharomyces cerevisiae.” Yeast, 10, 1793-1808. A deletion fragment consisting of a selectable marker flanked by 5′ and 3′ flanking sequences of PHO80 was constructed. At transformation, the PHO80 ORF was exchanged for hygromycin B phosphotransferase (hph) by homologous recombination. The plasmid pAG32 Goldstein et al., containing the selectable marker hph (hygromycin B phosphotransferase) was used as template for the PCR based construction of the PHO80 deletion fragment. For the PHO80 region, primers were designed according to SGD and for the hph region according to Gritz and Davis (1983) “Plasmid encoded hygromycin B resistance: the sequence of hygromycin B phosphotransferase gene and its expression in Escherichia coli and Saccharomyces cerevisiae.” Gene. 25, 178-188.

Deletion of PHO80

Forward primer:
5′:CAGCGTATATTGGCTTTCCTTTAATCTAATGCCCCAAGCCCACATAC
GATTTAGGTGACAC-3′ APPENDED AS SEQ ID NO: 10
Backwards primer:
5′-GGAGTTCTCAAGCTCATCTCGAAGTGTTTTCTGTCGCTTATGAATAC
GACTCACTATAGGGTG-3′ APPENDED AS SEQ ID NO: 11

Underlined sequences complement hph in pAG32 (Goldstein and McCusker, 1999, Yeast 15:1541-1553) The rest are deleting sequences that complement regions flanking PHO80 (SGD). The plasmid pAG32 (Goldstein and McCusker, 1999), containing the selectable marker hygromycin B phosphotransferase (hph), was used as template. FW primer complements to non-coding strain. Starting at ˜483 bp upstream hph start (ATG) in pAG32 and 315 bp upstream PHO80 start. BW primer complements to coding strand. Oligo. (63 bp.) starts 311 bp downstream hph end in pAG32, and 267 bp downstream PHO80 end in S. cerevisiae.

The PCR mix consisted of Thermopol buffer, 0. 1 μg/pl acetylated BSA (Bovine Serum Albumin), 0.2 mM dNTPs, 0.5 μM of each primer and 2 units of VENT polymerase. To each reaction approximately 0,7 μg/ml template DNA was added. Following the PCR reaction, products were separated by agarose gel electrophoresis (0.7 % agarose in TBE buffer. 100V) and purified by QIAquick Gel Extraction Kit (QIAGEN).

Resulting PCR product used for deletion of PHO80 by homologous recombination:

CAGCGTATATTGGCTTTCCTTTAATCTAATGCCCCAAGCCCACATACGAT
TTAGGTGACACTATAGAACGCGGCCGCCAGCTGAAGCTTCGTACGCTGCA
GGTCGACGGATCCCCGGGTTAATTAAGGCGCGCCAGATCTGTTTAGCTTG
CCTCGTCCCCGCCGGGTCACCCGGCCAGCGACATGGAGGCCCAGAATACC
CTCCTTGACAGTCTTGACGTGCGCAGCTCAGGGGCATGATGTGACTGTCG
CCCGTACATTTAGCCCATACATCCCCATGTATAATCATTTGCATCCATAC
ATTTTGATGGCCGCACGGCGCGAAGCAAAAATTACGGCTCCTCGCTGCAG
ACCTGCGAGCAGGGAAACGCTCCCCTCACAGACGCGTTGAATTGTCCCCA
CGCCGCGCCCCTGTAGAGAAATATAAAAGGTTAGGATTTGCCACTGAGGT
TCTTCTTTCATATACTTCCTTTTAAAATCTTGCTAGGATACAGTTCTCAC
ATCACATCCGAACATAAACAACCATGGGTAAAAAGCCTGAACTCACCGCG
ACGTCTGTCGAGAAGTTTCTGATCGAAAAGTTCGACAGCGTCTCCGACCT
GATGCAGCTCTCGGAGGGCGAAGAATCTCGTGCTTTCAGCTTCGATGTAG
GAGGGCGTGGATATGTCCTGCGGGTAAATAGCTGCGCCGATGGTTTCTAC
AAAGATCGTTATGTTTATCGGCACTTTGCATCGGCCGCGCTCCCGATTCC
GGAAGTGCTTGACATTGGGGAATTCAGCGAGAGCCTGACCTATTGCATCT
CCCGCCGTGCACAGGGTGTCACGTTGCAAGACCTGCCTGAAACCGAACTG
CCCGCTGTTCTGCAGCCGGTCGCGGAGGCCATGGATGCGATCGCTGCGGC
CGATCTTAGCCAGACGAGCGGGTTCGGCCCATTCGGACCGCAAGGAATCG
GTCAATACACTACATGGCGTGATTTCATATGCGCGATTGCTGATCCCCAT
GTGTATCACTGGCAAACTGTGATGGACGACACCGTCAGTGCGTCCGTCGC
GCAGGCTCTCGATGAGCTGATGCTTTGGGCCGAGGACTGCCCCGAAGTCC
GGCACCTCGTGCACGCGGATTTCGGCTCCAACAATGTCCTGACGGACAAT
GGCCGCATAACAGCGGTCATTGACTGGAGCGAGGCGATGTTCGGGGATTC
CCAATACGAGGTCGCCAACATCTTCTTCTGGAGGCCGTGGTTGGCTTGTA
TGGAGCAGCAGACGCGCTACTTCGAGCGGAGGCATCCGGAGCTTGCAGGA
TCGCCGCGGCTCCGGGCGTATATGCTCCGCATTGGTCTTGACCAACTCTA
TCAGAGCTTGGTTGACGGCAATTTCGATGATGCAGCTTGGGCGCAGGGTC
GATGCGACGCAATCGTCCGATCCGGAGCCGGGACTGTCGGGCGTACACAA
ATCGCCCGCAGAAGCGCGGCCGTCTGGACCGATGGCTGTGTAGAAGTACT
CGCCGATAGTGGAAACCGACGCCCCAGCACTCGTCCGAGGGCAAAGGAAT
AATCAGTACTGACAATAAAAAGATTCTTGTTTTCAAGAACTTGTCATTTG
TATAGTTTTTTTATATTGTAGTTGTTCTATTTTAATCAAATGTTAGCGTG
ATTTATATTTTTTTTCGCCTCGACATCATCTGCCCAGATGCGAAGTTAAG
TGCGCAGAAAGTAATATCATGCGTCAATCGTATGTGAATGCTGGTCGCTA
TACTGCTGTCGATTCGATACTAACGCCGCCATCCAGTGTCGAAAACGAGC
TCGAATTCATCGATGATATCAGATCCACTAGTGGCCTATGCGGCCGCGGA
TCTGCCGGTCTCCCTATAGTGAGTCGTATTCATAAGCGACAGAAAACACT
TCGAGATGAGCTTGAGAACTCC, appended as SEQ ID NO: 12

Nucleotides in bold show the ORF of the hgh gene. Underlined sequences shows the PCR primers, partly complementary to the PHO80 gene

The purified deletion fragment was transformed into YS18 by electroporation. The protcol for transformation was mainly based on a protocol at Gottschling Lab website. Transformants were incubated in 30° C. in 1 ml 1M sorbitol+1 ml 2×YPD for 2 h to allow expression of the drug resistance marker, then spread onto selective growth medium (YPD, 0.9 mg/ml hygromycin B). Colonies appeared after a few days and putative transformants were tested for accuracy by PCR. The resulting strain was designated YD80 (MATα; his3-11, 3-15; leu2-3, 2-112; ura3Δ5; canR; pho80Δ::hph)

2.5. Construction of combined pho80Δ deletion and PHO4 and PHO5 overexpressing strains.

The plasmid pYX212 containing the insert PHO5 (described above) was transformed into strain YD80 using a standard LiOAc S. cerevisiae transformation protocol. Furthermore, PHO4 was PCR cloned as described above using the primers:

Primers for Overexpression of PHO4

Forward primer (JP42)
5′-CGGAATTCATGGGCCGTACAACTTCTGAGG-3′,
APPENDED AS SEQ ID NO: 7
Backwards primers (JT42)
5′-CGCTCGAGTCACGTGCTCACGTTCTGCAG-3′,
APPENDED AS SEQ ID NO: 8

Underlined sequences complement EcoRI and XhoI restriction sites in pYX212 and the rest are sequences that complement start and end regions in PHO4 (SGD).

Sequence of pYX212 containing the insert PHO4:

GAATTCATGGGCCGTACAACTTCTGAGGGAATACACGGTTTTGTGGACGA
TCTAGAGCCCAAGAGCAGCATTCTTGATAAAGTCGCAGACTTTATCACCG
TAAACACGAAACGGCATGATGGGCGCGAGGACTTCAACGAGCAAAACGAC
GACCTGAACAGTCAAGAGAACCACAACAGCAGTGAGAATGGGAACCAGAA
TGAAAATGAACAAGACAGTCTCGCGTTGGACGACCTAGACCGCGCCTTTC
AGCTGGTGGAAGGTATGCATATCGACTGCATGATGCCCTCGCATGCGCAC
CACTCCCCAGCTACAACTGCTACAATCAAGCCGCGGCTATTATATTCGCC
GCTAATACACACGCAAACTGCGCTTCCCGTAACCATTTCGCCGAACTTGG
TCGCTACTGCTACTTCCACCACATCCGCTAACAAAGTCACTAAAAACAAG
AGTAATAGTAGTCCGTATTTGAACAAGCGCAGAGGTAAACCCGGGCCGGA
TTCGGCCACTTCGCTGTTCGAATTGCCCGACAGCGTTATCCCAACTCCGA
AACCGAAACCGAAACCAAAGCAATATCCGAAAGTTATTCTGCCGTCGAAC
AGCACAAGACGCGTATCACCGGTCACGGCCAAGACCAGCAGCAGCGCAGA
AGGCGTGGTCGTAGCAAGTGAGTCTCCTGTAATCGCGCCGCACGGATCGA
GCCATTCGCGGTCGCTGAGTAAGCGACGGTCATCGGGCGCGCTCGTGGAC
GATGACAAGCGCGAATCACACAAGCATGCAGAGCAAGCACGGCGTAATCG
ATTAGCGGTCGCGCTGCACGAACTGGCGTCTTTAATCCCCGCGGAGTGGA
AACAGCAAAATGTGTCGGCCGCGCCGTCCAAAGCGACCACCGTGGAGGCG
CCTGCCGGTACATCCGTCACCTACAGCAGAACGTGAGCACGTGACTCGAG
CTCAGCTAGCTAACTGAATAAGGAACAATGAACGTTTTTCCTTTCTCTTG
TTCCTAGTATTAATGACTGACCGATACATCCCTTTTTTTTTTTGTCTTTG
TCTAGCTCCAGCTTTTGTTCCCTTTAGTGAGGGTTAATTCAATTCACTGG
CCGTCGTTTTACAACGTCGTGACTGGGAAAACCCTGGCGTTACCCAACTT
AATCGCCTTGCAGCACATCCCCCTTTCGCCAGCTGGCGTAATAGCGAAGA
GGCCCGCACCGATCGCCCTTCCCAACAGTTGCGCAGCCTGAATGGCGAAT
GGCGCGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAG
CTCTAAATCGGGGGCTCCCTTTAGGGTTCCGATTTAGTGGTTTACGGCAC
CTCGACCCCAAAAAACTTGATTAGGGTGATGGTTCACGTAGTGGGCCATC
GCCCTGATAGACGGTTTTTCGCCCTTTGACGTTGGAGTCCACGTTCTTTA
ATAGTGGACTCTTGTTCCAAACTGGAACAACACTCAACCCTATCTCGGTC
TATTCTTTTGATTTATAAGGGATTTTGCCGATTTCGGCCTATTGGTTAAA
AAATGAGCTGATTTAACAAAAATTTAACGCGAATTTTAACAAAATATTAA
CGTTTACAATTTCCTGATGCGGTATTTTCTCCTTACGCATCTGTGCGGTA
TTTCACACCGCATAGGGTAATAACTGATATAATTAAATTGAAGCTCTAAT
TTGTGAGTTTAGTATACATGCATTTACTTATAATACAGTTTTTTAGTTTT
GCTGGCCGCATCTTCTCAAATATGCTTCCCAGCCTGCTTTTCTGTAACGT
TCACCCTGTACCTTAGCATCCCTTCCCTTTGCAAATAGTCCTCTTCCAAC
AATAATAATGTCAGATCCTGTAGAGACCACATCATCCACGGTTCTATACT
GTTGACCCAATGCGTCTCCCTTGTCATCTAAACCCACACCGGGTGTCATA
ATCAACCAATCGTAACCTTCATCTCTTCCACCCATGTCTCTTTGAGCAAT
AAAGCCGATAACAAAATCTTTGTCGCTCTTCGCAATGTCAACAGTACCCT
TAGTATATTCTCCAGTAGATAGGGAGCCCTTGCATGACAATTCTGCTAAC
ATCAAAAGGCCTCTAGGTTCCTTTGTTACTTCTTCTGCCGCCTGCTTCAA
ACCGCTAACAATACCTGGCCCCAGCACACCGTGTGCATTCGTAATGTCTG
CCCATTCTGCTATTCTGTATACACCCGCAGAGTACTGCAATTTGACTGTA
TTACCAATGTCAGCAAATTTTCTGTCTTCGAAGAGTAAAAAATTGTACTT
GGCGGATAATGCCTTTAGCGGCTTAACTGTGCCCTCCATCGAAAAATCAG
TCAATATATCCACATGTGTTTTTAGTAAACAAATTTTGGGACCTAATGCT
TCAACTAACTCCAGTAATTCCTTGGTGGTACGAACATCCAATGAAGCACA
CAAGTTTGTTTGCTTTTCGTGCATGATATTAAATAGCTTGGCAGCAACAG
GACTAGGATGAGTAGCAGCACGTTCCTTATATGTAGCTTTCGACATGATT
TATCTTCGTTTCCTGCAGGTTTTTGTTCTGTGCAGTTGGGTTAAGAATAC
TGGGCAATTTCATGTTTCTTCAACACTACATATGCGTATATATACCAATC
TAAGTCTGTGCTCCTTCCTTCGTTCTTCCTTCTGTTCGGAGATTACCGAA
TCAAAAAAATTTCAAAGAAACCGAAATCAAAAAAAAGAATAAAAAAAAAA
TGATGAATTGAATTGAAAAGCTGTGGTATGGTGCACTCTCAGTACAATCT
GCTCTGATGCCGCATAGTTAAGCCAGCCCCGACACCCGCCAACACCCGCT
GACGCGCCCTGACGGGCTTGTCTGCTCCCGGCATCCGCTTACAGACAAGC
TGTGACCGTCTCCGGGAGCTGCATGTGTCAGAGGTTTTCACCGTCATCAC
CGAAACGCGCGAGACGAAAGGGCCTCGTGATACGCCTATTTTTATAGGTT
AATGTCATGATAATAATGGTTTCTTAGACGTGCGGCCGCTCTAGAACTAG
TGGATCAATTCCACGGACTATAGACTATACTAGTATACTCCGTCTACTGT
ACGATACACTTCCGCTCAGGTCCTTGTCCTTTAACGAGGCCTTACCACTC
TTTTGTTACTCTATTGATCCAGCTCAGCAAAGGCAGTGTGATCTAAGATT
CTATCTTCGCGATGTAGTAAAACTAGCTAGACCGAGAAAGAGACTAGAAA
TGCAAAAGGCACTTCTACAATGGCTGCCATCATTATTATCCGATGTGACG
CTGCAGCTTCTCAATGATATTCGAATACGCTTTGAGGAGATACAGCCTAA
TATCCGACAAACTGTTTTACAGATTTACGATCGTACTTGTTACCCATCAT
TGAATTTTGAACATCCGAACCTGGGAGTTTTCCCTGAAACAGATAGTATA
TTTGAACCTGTATAATAATATATAGTCTAGCGCTTTACGGAAGACAATGT
ATGTATTTCGGTTCCTGGAGAAACTATTGCATCTATTGCATAGGTAATCT
TGCACGTCGCATCCCCGGTTCATTTTCTGCGTTTCCATCTTGCACTTCAA
TAGCATATCTTTGTTAACGAAGCATCTGTGCTTCATTTTGTAGAACAAAA
ATGCAACGCGAGAGCGCTAATTTTTCAAACAAAGAATCTGAGCTGCATTT
TTACAGAACAGAAATGCAACGCGAAAGCGCTATTTTACCAACGAAGAATC
TGTGCTTCATTTTTGTAAAACAAAAATGCAACGCGAGAGCGCTAATTTTT
CAAACAAAGAATCTGAGCTGCATTTTTACAGAACAGAAATGCAACGCGAG
AGCGCTATTTTACCAACAAAGAATCTATACTTCTTTTTTGTTCTACAAAA
ATGCATCCCGAGAGCGCTATTTTTCTAACAAAGCATCTTAGATTACTTTT
TTTCTCCTTTGTGCGCTCTATAATGCAGTCTCTTGATAACTTTTTGCACT
GTAGGTCCGTTAAGGTTAGAAGAAGGCTACTTTGGTGTCTATTTTCTCTT
CCATAAAAAAAGCCTGACTCCACTTCCCGCGTTTACTGATTACTAGCGAA
GCTGCGGGTGCATTTTTTCAAGATAAAGGCATCCCCGATTATATTCTATA
CCGATGTGGATTGCGCATACTTTGTGAACAGAAAGTGATAGCGTTGATGA
TTCTTCATTGGTCAGAAAATTATGAACGGTTTCTTCTATTTTGTCTCTAT
ATACTACGTATAGGAAATGTTTACATTTTCGTATTGTTTTCGATTCACTC
TATGAATAGTTCTTACTACAATTTTTTTGTCTAAAGAGTAATACTAGAGA
TAAACATAAAAAATGTAGAGGTCGAGTTTAGATGCAAGTTCAAGGAGCGA
AAGGTGGATGGGTAGGTTATATAGGGATATAGCACAGAGATATATAGCAA
AGAGATACTTTTGAGCAATGTTTGTGGAAGCGGTATTCGCAATATTTTAG
TAGCTCGTTACAGTCCGGTGCGTTTTTGGTTTTTTGAAAGTGCGTCTTCA
GAGCGCTTTTGGTTTTCAAAAGCGCTCTGAAGTTCCTATACTTTCTAGAG
AATAGGAACTTCGGAATAGGAACTTCAAAGCGTTTCCGAAAACGAGCGCT
TCCGAAAATGCAACGCGAGCTGCGCACATACAGCTCACTGTTCACGTCGC
ACCTATATCTGCGTGTTGCCTGTATATATATATACATGAGAAGAACGGCA
TAGTGCGTGTTTATGCTTAAATGCGTACTTATATGCGTCTATTTATGTAG
GATGAAAGGTAGTCTAGTACCTCCTGTGATATTATCCCATTCCATGCGGG
GTATCGTATGCTTCCTTCAGCACTACCCTTTAGCTGTTCTATATGCTGCC
ACTCCTCAATTGGATTAGTCTCATCCTTCAATGCTATCATTTCCTTTGAT
ATTGGATCATATGCATAGTACCGAGAAACTAGTGCGAAGTAGTGATCAGG
TATTGCTGTTATCTGATGAGTATACGTTGTCCTGGCCACGGCAGAAGCAC
GCTTATCGCTCCAATTTCCCACAACATTAGTCAACTCCGTTAGGCCCTTC
ATTGAAAGAAATGAGGTCATCAAATGTCTTCCAATGTGAGATTTTGGGCC
ATTTTTTATAGCAAAGATTGAATAAGGCGCATTTTTCTTCAAAGCTGCGG
CCGCACTCTCACTAGTACGTCAGGTGGCACTTTTCGGGGAAATGTGCGCG
GAACCCCTATTTGTTTATTTTTCTAAATACATTCAAATATGTATCCGCTC
ATGAGACAATAACCGTGATAAATGCTTCAATAATATTGAAAAAGGAAGAG
TATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAA
CAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGAC
GCCGGGCAAGAGCAACTCGCTCGCCGCATACACTATTCTCAGAATGACTT
GGTTGAGTACTCACCAGTCACAGAAAAGCATCTTACGGATGGCATGACAG
TAAGAGAATTATGCAGTGCTGCCATAACCATGAGTGATAACACTGCGGCC
AACTTACTTCTGACAACGATCGGAGGACCGAAGGAGCTAACCGCTTTTTT
GGACAACATGGGGGATCATGTAACTCGCCTTGATCGTTGGGAACCGGAGC
TGAATGAAGCCATACCAAACGACGAGCGTGACACCACGATGCCTGTAGCA
ATGGCAACAACGTTGCGCAAACTATTAACTGGCGAACTACTTACTCTAGC
TTCCCGGCAACAATTAATAGACTGGATGGAGGCGGATAAAGTTGCAGGAC
CACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAA
CTATGGATGAACGAAATAGACAGATCGCTGAGATAGGTGCCTCACTGATT
AAGCATTGGTAACTGTCAGACCAAGTTTACTCATATATACTTTAGATTGA
TTTAAAACTTCATTTTTAATTTAAAAGGATCTAGGTGAAGATCCTTTTTG
ATAATCTCATGACCAAAATCCCTTAACGTGAGTTTTCGTTCCACTGAGCG
TCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTT
AGGCCACCACTTCAAGAACTCTGTAGCACCGCCTACATACCTCGCTCTGC
TAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTG
AACGGGGGGTTCGTGCACACAGCCCAGCTTGGAGCGAACGACCTACACCG
AACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCC
CTGATTCTGTGGATAACCGTATTACCGCCTTTGAGTGAGCTGATACCGGT
CGCCGCAGCCGAACGACCGAGCGCAGCGAGTCAGTGAGCGAGGAAGCGGA
AGAGCGCCCAATACGCAAACCGCCTCTCCCCGCGCGTTGGCCGATTCATT
AATGCAGCTGGCACGACAGGTTTCCCGACTGGAAAGCGGGCAGTGAGCGC
AACGCAATTAATGTGAGTTACCTCACTCATTAGGCACCCCAGGCTTTACA
CTTTATGCTTCCGGCTCCTATGTTGTGTGGAATTGTGAGCGGATAACAAT
TTCACACAGGAAACAGCTATGACCATGATTACGCCAAGCTCGAAATACGA
CTCACTATAGGGCGAATTGGGTACCGGGCCGGCCGTCGAGCTTGATGGCA
TCGTGGTGTCACGCTCGTCGTTTGGTATGGCTTCATTCAGCTCCGGTTCC
CAACGATCAAGGCGAGTTACATGATCCCCCATGTTGTGAAAAAAAGCGGT
TAGCTCTTCGGTCCTCCGATCGTTGTCAGAAGTAAGTTGGCCGCAGTGTT
ATCACTCATGGTTATGGCAGGAACTGCATAATTCTCTTACTGTCATGCCA
TCCGTAAGATGCTTTTCTGTGACTGGTGTACTCAACCAAGTCATTCTGAG
AATAGTGTATGCGGCGACCGAGTTGCTCTTGCCCGGCGTCAACACGGGAT
AATACCGCGCCACATAGCAGAACTTTAAAAGTGCTCATCATTGGAAAACG
TTCTTCGGGGCGAAAACTCTCAAGGATCTTACCGCTGTTGAGATCCAGTT
CGATGTAACCCACTCGTGCACCCAACTGATCTTCAGCATCTTTTACTTTC
ACCAGCGTTTCTGGGTGAGCAAAAACAGGAAGGCAAAATGCCGCAAAAAA
GGGAATAAGGGCGACACGGAAATGTTGAATACTCATACTCTTCCTTTTTC
AATATTATTGAAGCATTTATCAGGGTTATTGTCTCATGAGCGATACATAT
TTGAATGTATTTAGAAAAATAAACAAATAGGGGTTCCGCGCACATTTCCC
CGAAAAGTGCCACCTGACGTCTAAGAAACCATTATTATCATGACATTAAC
CTATAAAAATAGGCGTATCACGAGGCCCTTTCGTCTTCAAGAATTGGGGA
TCTACGTATGGTCATTCTTCTTCAGATTCCCTCATGGAGAAGTGCGGCAG
ATGTATATGACAGAGTCGCCAGTTTCCAAGAGACTTTATTCAGGCACTTC
CATGATAGGCAAGAGAGAAGACCCAGAGATGTTGTTGTCCTAGTTACACA
TGGTATTTATTCCAGAGTATTCCTGATGAAATGGTTTAGATGGACATACG
AAGAGTTTGAATCGTTTACCAATGTTCCTAACGGGAGCGTAATGGTGATG
GAACTGGACGAATCCATCAATAGATACGTCCTGAGGACCGTGCTACCCAA
ATGGACTGATTGTGAGGGAGACCTAACTACATAGTGTTTAAAGATTACGG
ATATTTAACTTACTTAGAATAATGCCATTTTTTTGAGTTATAATAATCCT
ACGTTAGTGTGAGCGGGATTTAAACTGTGAGGACCTCAATACATTCAGAC
ACTTCTGACGGTATCACCCTACTTATTCCCTTCGAGATTATATCTAGGAA
CCCATCAGGTTGGTGGAAGATTACCCGTTCTAAGACTTTTCAGCTTCCTC
TATTGATGTTACACTCGGACACCCCTTTTCTGGCATCCAGTTTTTAATCT
TCAGTGGCATGTGAGATTCTCCGAAATTAATTAAAGCAATCACACAATTC
TCTCGGATACCACCTCGGTTGAAACTGACAGGTGGTTTGTTACGCATGCT
AATGCAAAGGAGCCTATATACCTTTGGCTCGGCTGCTGTAACAGGGAATA
TAAAGGGCAGCATAATTTAGGAGTTTAGTGAACTTGCAACATTTACTATT
TTCCCTTCTTACGTAAATATTTTTCTTTTTAATTCTAAATCAATCTTTTT
CAATTTTTTGTTTGTATTCTTTTCTTGCTTAAATCTATAACTACAAAAAA
CACATACAG Appended as Seq ID: 9

Bolded sequence shows PH04
Underlined sequences represent sites for primers.

Also pYX212 with PHO4 was transformed into YD80 using the methods already described.

Sample Preparation, Inositol Phosphates and pNPP

1.5 ml of cell suspension was withdrawn at intervals during the growth and centrifuged for 3 minutes at 13000 rpm to pellet the cells. One ml of the supernatant was transferred to a new micro-tube and acidified by adding 150 μl of 4 M HCl. This yields approximately 0.5 M HCl, which is enough to inactivate all enzymatic activity potentially left in the supernatant. Chemical acid hydrolysis of inositol phosphates does not take place in such solutions without strong heating. These samples were stored in freezer until use for analysis of IP:s and pNPP. A reference sample for identification of peaks was prepared by chemically hydrolyzing 1 mM sodium phytate (Na-IP6) in 2 ml of 0.5 M HCl. The solution was heated to 110° C. for 15 h yielding a mixture of isomers of IP:s that was stored in freezer until used as reference sample.

Bread Dough Making, Phosphate Extraction and Analysis

To analyze the Pᵢ and IP6 content in a typical environment, baker's yeast are exposed to during bread leavening two simple doughs were mixed, in which two types of flour were used: whole meal wheat flour (100% extraction rate; i.e. the proportion of the whole wheat grain obtained as finished flour), and normal wheat flour (extraction rate 60%). The dough mixture contained: 300 g flour, log sucrose, 3 g NaCl, 20 g commercial baker's yeast (Swedish Yeast Company) and 200 g tap-water. At time zero and thereafter every 15 min, 20 g of dough was withdrawn and mixed with 40 ml 0.5 M HCl (at room temperature this HCl concentration will not hydrolyze IP6, but is sufficient to inactivate enzymatic activity). Inorganic phosphate (Pᵢ) and IP6 were extracted by magnetically stirring the mixture for three hours at room temperature. The extracts were centrifuged, the supernatant collected and frozen until use. For analysis, the samples were thawed and 1 ml was centrifuged in a micro-tube for 5 min at 13000×g. The resulting supernatants were appropriately diluted with mQ-water and inositol phosphates were analyzed by HPIC chromatography as described in previous paragraph. The ion chromatographic analysis of Pᵢ were performed using a Dionex (Sunnyvale, Calif., USA) model 4500i equipped with a 50 μl loop injector, PAX-100 guard and analytical column, Anion Micro Membrane Supressor and a conductivity detector. The samples were eluted with a linear gradient of water and increasing portion of 200 mM NaOH, starting with 6% NaOH and ending after 35 min at 50% NaOH

Results

Expression and Repression of Phytase Activity in Wild-Type Yeasts

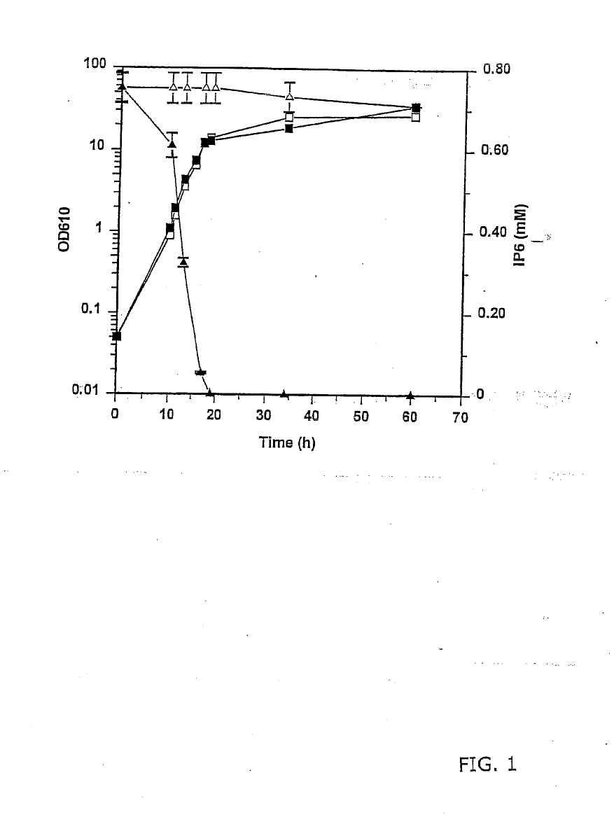

IP6 is efficiently degraded by yeast in a synthetic medium with IP6 as the sole phosphorous source. Tüirk et al. (2000) Inositol hexaphosphate hydrolysis by baker's yeast. Capacity, kinetics, and degradation products. J. Agric. Food Chem. 48, 100-104 In order to test whether extracellular IP6 hydrolysis by S. cerevisiae was repressed by inorganic phosphate (Pᵢ), synthetic yeast minimal medium containing Pᵢ (26 mM) was supplemented with IP6 (CBSIP6+Pᵢ). For all S. cerevisiae tested, that is SKQ2n, CBS 7764, CBS 7765, YS18, BY4741 and YMR4 virtually no breakdown of IP6 was detected in the IP6+Pᵢ cultures, showing an efficient repression of phytase activity by high levels of extracellular Pᵢ. Referring to FIG. 1, growth of S. cerevisiae SKQ2n (squares) and extracellular concentration of inositol hexaphosphate (IP6; triangles), measured by HPIC, as a function of time is shown. Growth was performed in synthetic CBS medium with 2% (wt/vol) glucose as carbon and energy source. Filled symbols: Inositol hexaphosphate (IP6) as the sole phosphorous source; open symbols: inositol hexaphosphate plus inorganic phosphate (Pᵢ) as combined phosphorous sources. Data are from a representative experiment performed many times. Error bars on IP6 data show maximum variation between repeated HPIC analysis. The typical pattern for the IP6 concentration in the medium during growth in the absence and presence of Pᵢ is depicted for strain SKQ2n in FIG. 1.

In this experiment, all the IP6 was depleted before 20 h of growth in the CBSIP6 culture, while in the CBSIP6+Pᵢ culture no IP6 was degraded at that time. For all strains, the growth rates, as calculated by regression in the exponential respiro-fermentative phase (glucose growth), were as rapid in CBSIP6 medium as compared with CBSIP6+Pᵢ or CBSPᵢ media, showing efficient utilization of IP6 as P-source for growth by S. cerevisiae.

The same type of experiments were performed also in complex and rich YPD medium, either with the addition of only IP6 (YPDIP6) or with the addition of IP6 and Pi (YPDIP6+Pᵢ). The YPD experiments yielded data consistent with the CBS cultures, that is IP6 degradation without loss in growth rate in YPDIP6 medium and repression of phytase activity in YPDIP6+Pᵢ medium (data not shown). However, the Pᵢ induced repression was for some strains (e.g. YS18) less pronounced in YPD as compared with CBS medium, that is a certain constitutive, not negligible, phytase activity was detected.

Inositol Phosphates Formed During IP6 Degradation

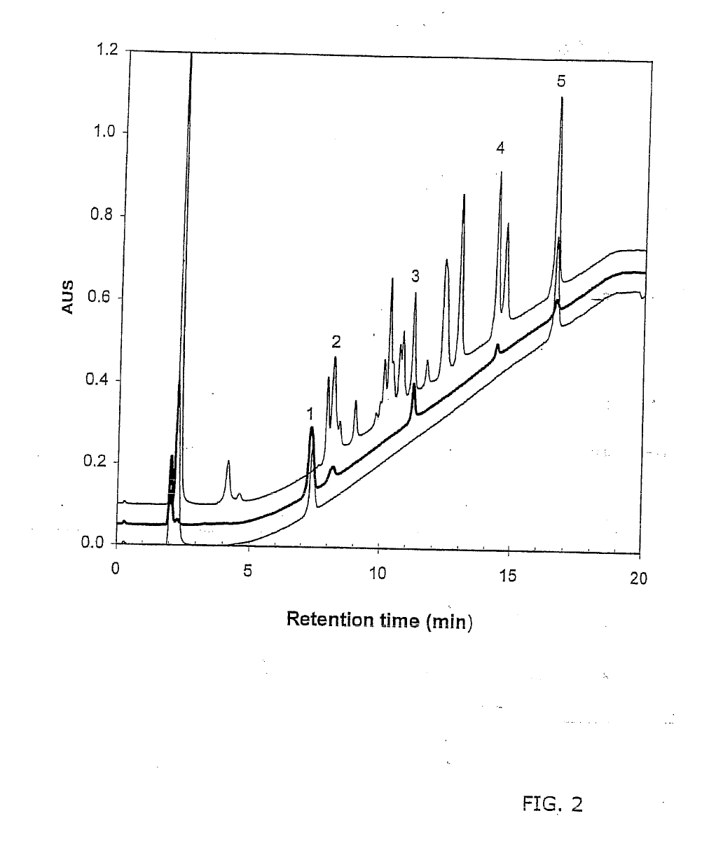

IP6 and the breakdown products formed as a result of IP6 hydrolysis by the yeast were identified and quantified by HPIC. Referring to FIG. 2, chromatographic profiles (HPIC) of chemical acid hydrolysate of sodium phytate (first line), and supernatant from S. cerevisiae cultures (second and third lines) after 15 h of growth is shown. S. cerevisiae was cultured in CBS medium containing either IP6 as the sole P-source (second line, thick) demonstrating yeast phytase activity, or in CBS medium with IP6 and Pᵢ as combined P-sources (third line) showing Pᵢ-induced repression of phytase activity. Numbered peaks: 1: SO₄⁻²; 2: Ins(1,2,6)P₃; 3: Ins(1,2,5,6)P₄; 4: Ins(1,2,4,5,6)P₅; 5: IP6.

The chromatographic profile showed that the enzymes exerting the yeast phytase activity are very specific with respect to position on the inositol ring. Only one isomer of each IP₅, IP₄ and IP₃ was detected for samples withdrawn during growth of S. cerevisiae in CBSIP6 medium (FIG. 2, second line). These isomers aligned with an acid chemical hydrolysate (FIG. 2, first line, in which all peaks previously have been identified) proved to be I(1,2,4,5,6)P₅, I(1,2,5,6)P₄, I(1,2,6)P₃, respectively. It is known that peak 2 in the hydrolysate may contain a mixture of I(1,2,6)P₃ and I(1,2,3)P₃. However, the I(1,2,3)P₃ isomer can be excluded in the yeast samples since position 3 was already missing. The same pattern of IP isomers was obtained for all strains tested in our collection, including laboratory strains and several wild-types. The lower, third line in FIG. 2 shows the extracellular composition of IP:s from a S. cerevisiae CBSIP6+Pi culture at 15 hrs of growth. In such cultures no lower inositol phosphates were detectable and the concentration of IP6 (peak 5) remained high through out the experiment proving the Pᵢ induced repression of extracellular phytase activity.

PHO Deletion Mutants

The major secretory acid phosphatase, encoded by PHO5, may be involved in phytate hydrolysis. A PHO5 deletion mutant may be less efficient in degrading extracellular IP6 as compared with the corresponding parent strain. For this purpose the pho3Δpho5Δ double mutant YMR4 was used. By using this mutant the effect of missing PHO3, in addition to PHO5, was assessed. However, this gene may be less important since it has been shown to be repressed by thiamine, (which is present in the CBS medium) and its product is mainly a thiamine phosphatase, Praekelt et al. (1994) Regulation of TH14 (MOL1), a thiamine-biosynthetic gene of Saccharomyces cerevisiae. Yeast. 10, 481-490. Contrary to expectation, the mutant was not affected.

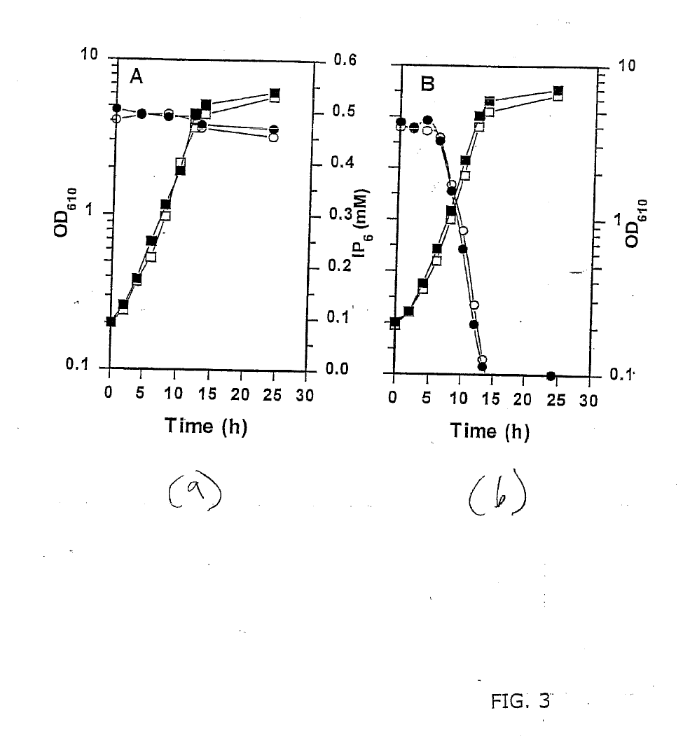

Referring to FIGS. 3A and 3B, growth (squares) of S. cerevisiae parent strain YS18 (open symbols) and S. cerevisiae YMR4 pho5Δpho3Δ double deletion mutant (filled symbols) and extracellular concentration of IP6 (circles) as determined by HPIC. FIG. 3A shows synthetic CBS medium with glucose as carbon and energy source and IP6 plus Pᵢ as phosphorous sources (repressing conditions). FIG. B shows synthetic CBS medium with glucose as carbon and energy source and IP6 as the sole phosphorous source (de-repressing conditions). Represented data are means of double samples from a representative experiment run twice.

In repeated experiments strain YMR4 hydrolyzed extracellular IP6 at a rate equal to its parent strain YS18 (FIG. 3B). In addition, FIG. 3A shows that in CBSIP6 +Pᵢ medium (repressing conditions), the pattern of growth and the Pᵢ induced repression was unaffected in the mutant strain YMR4, which closely follows the pattern for YS18. For both strains at time 24 h after inoculation, 94% of the initial amount of IP6 was still present in the CBSIP6+Pᵢ cultures (FIG. 3A). At this time, neither IP6 nor any other lower IP:s were detectable in the medium of the parallel CBSIP6 culture (FIG. 3B). Additional evidence for independence of PHO5 in this context was provided by the rate of growth for the mutant in CBSIP6 medium. The calculated growth rate in the exponential respiro-fermentative phase (growth on glucose, CBSIP6 medium) was 0.33 h⁻¹ (generation time of 2.1 h) for both YMR4 and its parent strain YS18. This is not evidence that Pho5p and Pho3p are unable to hydrolyze IP6, it is only evidence that shows that they are not needed by the yeast for intact phytase activity.

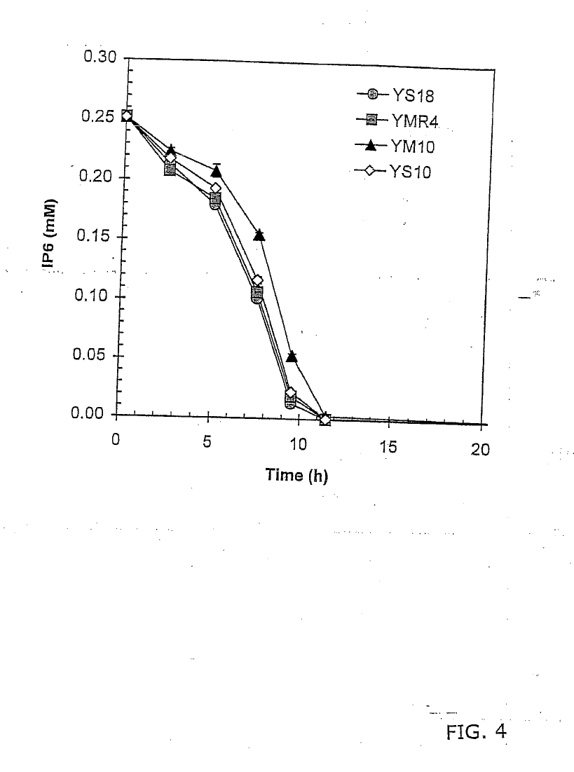

By PCR and homologous recombination PHO10 was deleted in YMR4 and YS18 thereby obtaining a pho5Δpho3Δpho10Δ triple mutant and pho10Δ single mutant labeled YM10 and YS10, respectively. Referring to FIG. 4, concentration of inositol hexaphosphate (IP6) in yeast culture supernatant as a function of growth in CBS medium with IP6 as the sole phosphorous source is shown. Grey circles: YS18 parent strain with all PHO genes intact; filled squares: YMR4 pho3ΔApho5Δ double mutant; filled triangles: YM10 pho3Δpho5Δpho10Δ triple mutant; and open diamonds: YS10 pho10Δ single deletion mutant. Data are means of double samples from separate cultures and error bars show the variation between those. These strains were cultured in CBSIP6 and YPDIP6 medium, and the medium concentration of IP6 was monitored by HPIC during growth. In CBS, the rate of IP6 degradation was unaffected in YS1O showing that also PHO10 was superfluous (FIG. 4) for intact phytase activity.

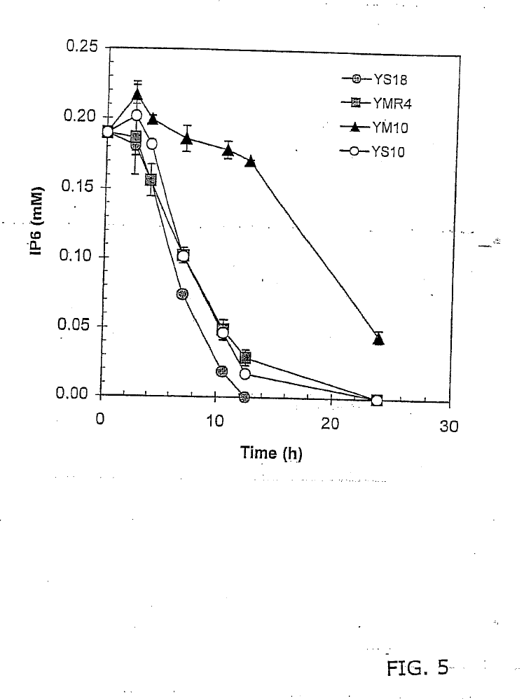

Even in YM10, in which the only remaining secretory phosphatase is Pho11p, the net extracellular phytase activity was unaffected as compared with YS18 and YMR4. Only a slight, but significant, decrease in the rate of IP6 degradation was in CBS detected in this mutant (FIG. 4). However, in the complex medium YPD, the triple mutant showed a strong reduction in rate of extracellular IP6 (FIG. 5) with maintained growth rate, suggesting phytase activity for Pho10p and at least one more enzyme.

Referring to FIG. 5, concentration of inositol hexaphosphate (IP6) in yeast culture supernatant as a function of growth in complex YPD medium with IP6 as the added phosphorous source is shown. Grey circles: YS18 parent strain with all PHO genes intact; filled squares: YMR4 pho3Δpho5Δ double mutant; filled triangles: YM10 pho3Δpho5Δpho10Δ triple mutant; and open diamonds: YS10 pho10Δ single deletion mutant. Data are means of double samples from separate cultures and error bars show the variation between those.

Overexpression of PHO5

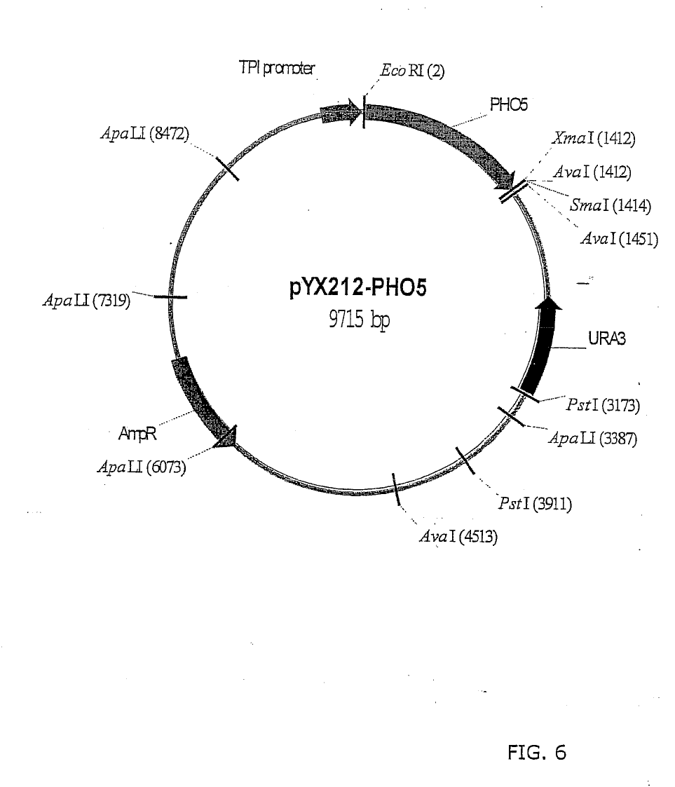

Referring to FIG. 6, a vector construct consisting of plasmid pYX212 containing the insert PHO5 used for transformation of YS18 and YMR4. The glycolytic promoter triose phosphate isomerase (TPI1) controlling expression of PHO5, the yeast selection marker URA3 and the Amp^{R} gene are depicted.

In order to assess the impact of exclusively PHO5 expression on the extracellular IP6 degradation YMR4 and YS18 were both transformed with pYX212 containing the insert PHO5 controlled by the constitutive glycolytic promoter TPI1 (FIG. 6).

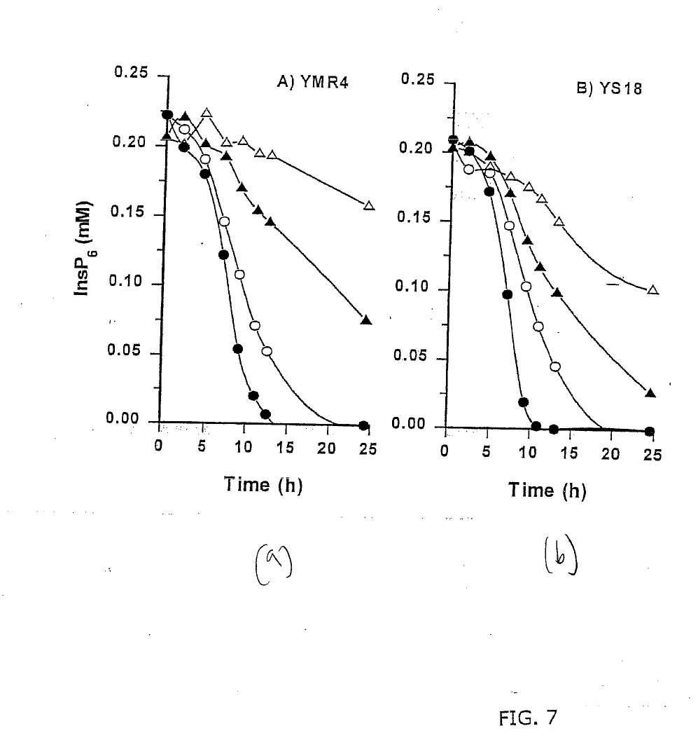

The resulting strains were designated YM-p5 and YS-p5, respectively (Table 1). The experiments with these mutants were performed in YPD medium with the addition of the appropriate phosphorous source. Growth and extracellular IP6 concentration were assessed as a function of time. Referring to FIGS. 7A and 7B, extracellular concentration of IP6 as a function of growth time of S. cerevisiae YMR4 (A) and YS18 (B), with and without the PHO5 containing plasmid pYX212. Open symbols: strains without plasmid; solid symbols: strains containing the plasmid pYX212-PHO5. The experiment was performed in YPD medium with 2% glucose supplemented with either only IP6 (circles) or with both IP6 and Pᵢ (triangles). Represented data are means from two to three independent experiments with a standard deviation never exceeding 5%. From both YM-p5 and YS-p5 data it is evident that PHO5 encodes an enzyme with phytase activity (FIGS. 7A and B).

Under normally repressing conditions (YPDIP6+Pi) IP6 was by YM-p5 and YS-p5 degraded at a fairly high rate (filled triangles in FIGS. 7A and 7B), demonstrating that phytase activity was in these mutants constitutively expressed. The corresponding parent strains lacking the plasmid are shown as controls (open triangles). These controls were in YPD somewhat lesser repressed as compared with CBS medium, however, much more repressed than the cells with plasmid. After 24 h of growth strain YM-p5 had degraded 63% of the initial IP6 whereas in the YMR4 control strain the corresponding value at 24 h was only 22.5% (FIG. 6). The rate of IP6 degradation in YPDIP6 non-repressing medium was also analyzed. During these conditions PHO10 and PHO11 plus the introduced PHO5 would be expressed in YM-p5, and PHO5, PHO10 and PHO11 plus the introduced PHO5 in the YS-p5 strain. The data show that the plasmid located PHO5 indeed increased the net rate of IP6 degradation (FIGS. 7A and 7B) in both strains. The effect was more pronounced in YS-p5, in which the PHO5 gene is present both in the genome and on the plasmid. The combined data from the PHO5 overexpression experiments and the experiments with deletion strains compared with their parent strains (FIGS. 3 and 4) shows that Pho5p, Pho10p and at least one more enzyme, believed to be Pho11p, are by definition all phytases.

Deletion of the Regulator Genes PHO80, PHO85 and PHO2

The PHO system is involved in the yeast phytase activity. Accordingly, deletions in the regulatory genes PHO80 and PHO85 yield constitutive phytase activity.

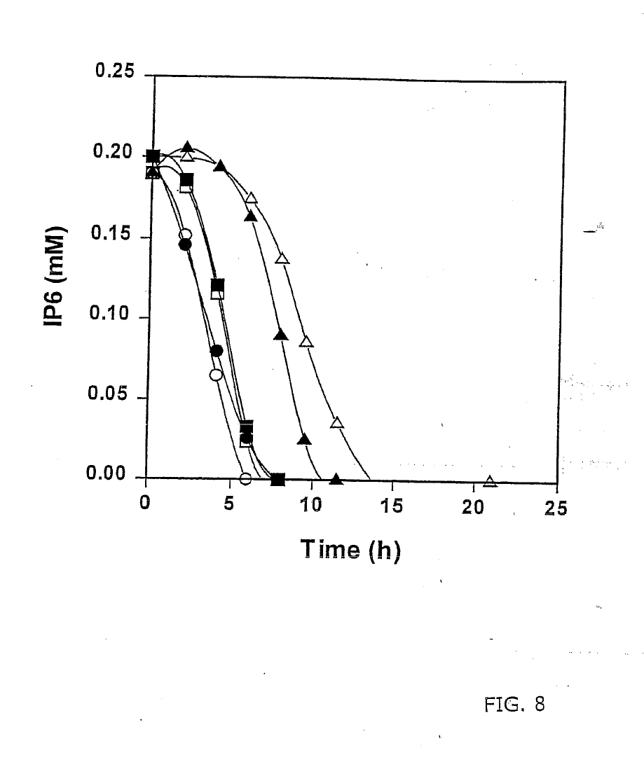

Referring to FIG. 8, a direct comparison of phytate degrading capacity between PHO5 overexpressing strains, and pho80Δ and pho85Δ deletions mutants. Extracellular concentration of IP6 is plotted as a function of growth time of S. cerevisiae YS18-p5 (triangles) with the PHO5 containing plasmid pYX212, S. cerevisiae pho80Δ deletion mutant (squares) and pho85Δ deletion mutant (circles) is shown. The experiment was performed in YPD medium with 2% glucose supplemented with either only IP6 (solid symbols) or with both IP6 and Pᵢ (open symbols). Data are means from two independent experiments with a standard deviation never exceeding 5%.

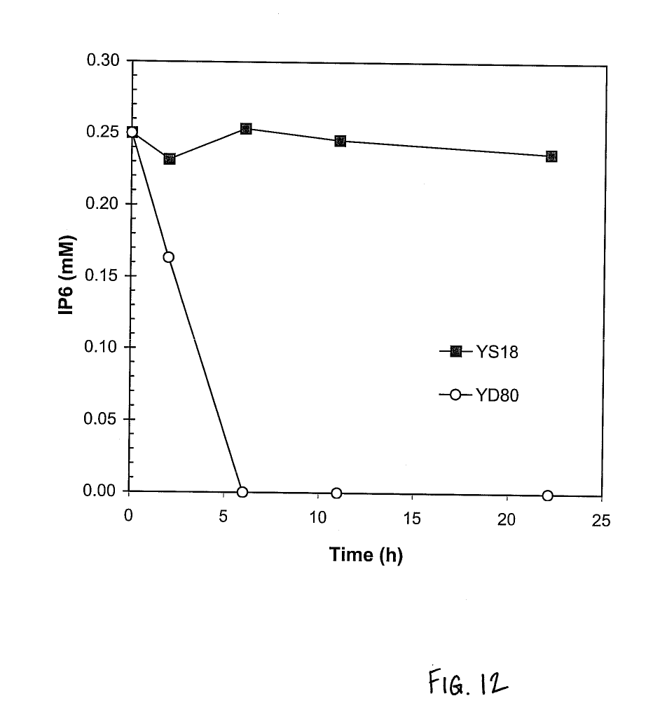

As shown in FIG. 8 these mutants were highly effective in degrading IP6, and completely constitutive demonstrating that the PHO system indeed is involved in S. cerevisiae phytase activity. Since the net rate of IP6 degradation was very rapid, these strains were compared with the YS18-p5 (PHO5 overexpressing) strain. Both strain pho80Δ and pho85Δ showed a higher net rate in IP6 degradation as compared with the YS18-p5 (FIG. 8). The growth rate was unchanged in strain pho80Δ, however significantly reduced in strain pho85Δ. In addition to the pho80Δ and pho85Δ strains retrieved from BY4741 (EUROSCARF collection), another pho80Δ, referring to FIG. 12, mutant was constructed by deletion in YS18, yielding strain YD80. The highly efficient extracellular phytase activity was reconfirmed in this strain.

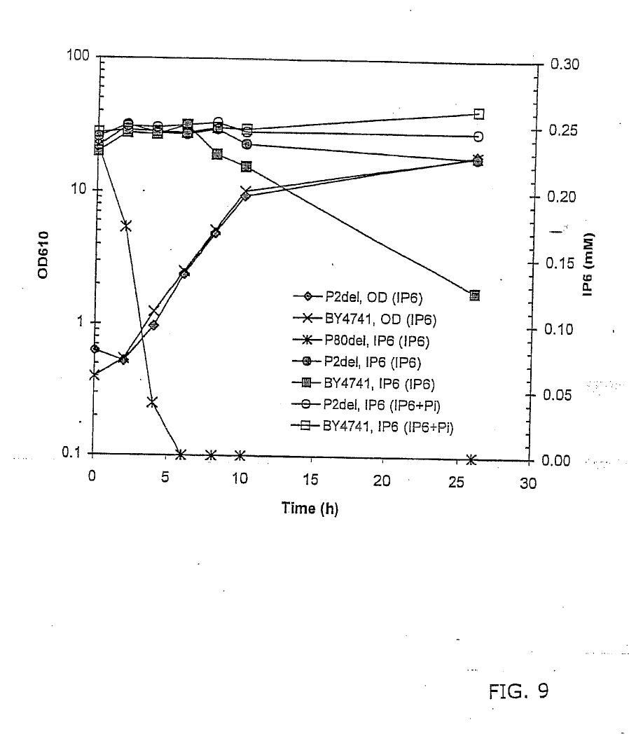

Referring to FIG. 9, phytate degrading capacity of pho2Δ deletion mutant is shown. For comparison, pho80Δ deletion strain and BY4741 with all PHO genes intact are also shown. Extracellular concentration of IP6 is plotted as a function of growth time in YPD. For explanation of symbols see Figure. The P-source used is shown within parenthesis. In addition to IP6 data, growth data as OD610, are shown for the pho2Δ deletion mutant and for its parent strain BY4741. Data are means from two independent experiments with a standard deviation never exceeding 5%.

PHO2 is together with PHO4 known to positively regulate the expression of the secretory phosphatases PHO5, PHO10 and PHO11. In a direct comparison the pho2 deletion mutant was demonstrated to lack virtually all phytase activity, demonstrating that yeast phytase activity is largely a matter of its PHO system. The phytase activity may therefore be further increased by overexpressing PHO2 and/or PHO4.

Expression of PHO3

The expression of PHO3, in addition to expression of PHO5, PHO10 and PHO11 may increase the rate of extracellular IP6 degradation. For this purpose the parent strain YS18 was used in a CBS medium excluding thiamine, which is known to repress PHO3 expression, Nosaka et al. (1989) A possible role for acid phosphatase with thiamin-binding activity encoded by PHO3 in yeast. FEMS Microbiol. Lett. 51, 55-59; Nosaka (1990) High affinity of acid phosphatase encoded by PHO3 gene in Saccharomyces cerevisiae for thiamin phosphates. Biochim. Biophys. Acta. 1037, 147-154. As controls normal CBSIP6 medium (with thiamine) was used, with and without thiamine phosphate to ensure that the yeast was not negatively affected by the missing thiamine. Growth rates and IP6 degradation were by YS18 not different between the three media used, indicating that PHO3 is of no or minor significance for yeast phytase activity (data not shown).

Competitive Degradation of IP6 and pNPP

The PHO system in S. cerevisiae has frequently been studied using phenyl phosphate and para-nitrophenyl phosphate (pNPP) as substrates. Monod et al. (1989) Functional analysis of the signal-sequence processing site ofyeast acid phosphatase. Eur. J. Biochem. 182, 213 -22 1; Shnyreva et al. (1996) Biochemical properties and excretion behavior of repressible acid phosphatases with altered subunit composition. Microbiol. Res. 151, 291-300; Martinez et al. (1998) Identification, cloning and characterization of a derepressible Na⁺-coupled phosphate transporter in Saccharomyces cerevisiae. Mol. Gen. Genet. 258, 628-638. These compounds are used as model organic phosphorous source. Yeasts are often exposed to several organic phosphorous sources.

Accordingly, the impact of adding a second organic phosphorous compound (PNPP) on the rate of IP6 degradation was studied. Both compounds are readily degraded by S. cerevisiae and since the PHO system is involved in both cases a competitive situation arose.

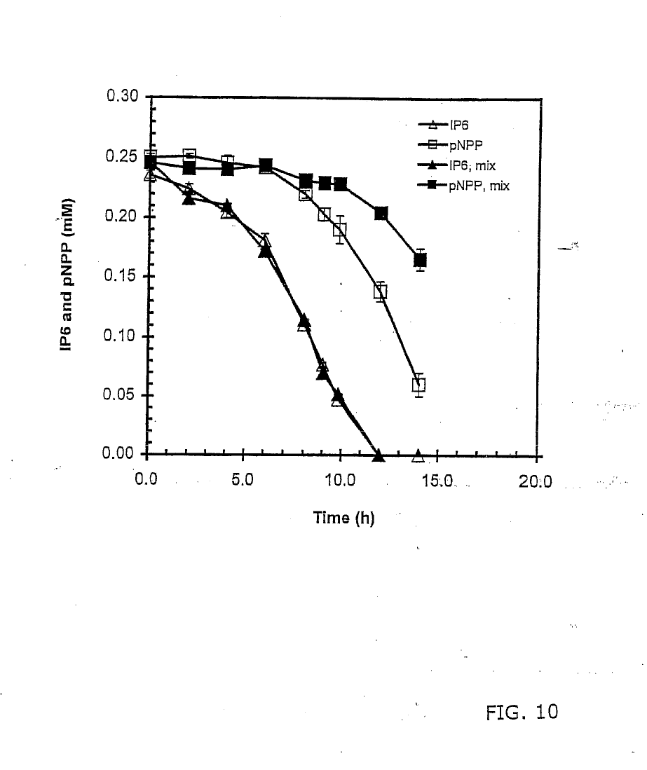

Referring to FIG. 10, extracellular concentration of para-nitrophenyl phosphate (PNPP, squares) and IP6 (triangles) as a function of growth of S. cerevisiae SKQ2n in CBS medium with 2% glucose as carbon and energy source is shown. The CBS medium was supplemented either with IP6 as the sole P-source (open triangles), with pNPP as the sole P-source (open squares) or with both IP6 and pNPP as combined P-sources (Solid symbols).

In the experiment, with both IP6 and pNPP present and Pᵢ absent, the rate of IP6 degradation was completely unchanged as compared with IP6 alone (FIG. 10). However, the rate of pNPP was delayed by presence of IP6 compared to pNPP alone. Most of the degradation of pNPP occurred after IP6 was depleted from the medium. At 12 h of growth, IP6 was depleted in both cultures, whereas only 17% of the initial pNPP was used in the mixed culture, and 49% in the sole pNPP culture. The experiment was also performed with the pNPP concentration set to 6 times the concentration of IP6 (2.25 mM) to obtain equal stoichiometry in number of available phosphate groups. Six times more frequently pNPP randomly met the enzymes. The experiment yielded similar data, that is no change in rate of IP6 hydrolysis in the presence of pNPP and delayed pNPP degradation as compared with pNPP alone was observed (data not shown).

Content of Inorganic Phosphate and IP6 in Bread Dough

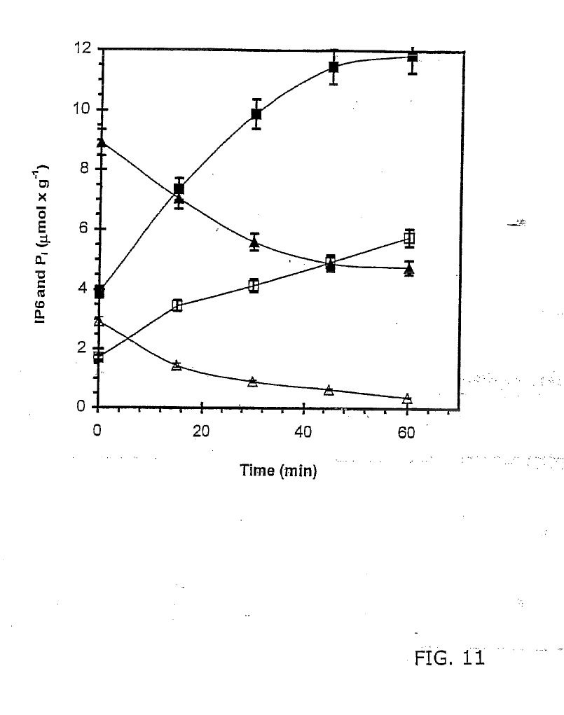

To understand the yeast environment during bread making, relative to the PHO system, the Pᵢ and IP6 content were analyzed in two types of wheat based bread doughs. In both doughs the level of Pᵢ increased and the level of IP6 decreased as a function of leavening time (FIG. 11).

Referring to FIG. 11, the content of inositol hexaphosphate (IP6, triangles) and inorganic phosphate (Pᵢ; squares), expressed as μmol per gram wet dough, as a function of leavening time in two types of wheat based doughs is shown. Solid symbols: whole meal wheat flour (100% extraction rate; i.e. the proportion of the whole wheat grain obtained as finished flour), and open symbols: white wheat flour (extraction rate 60%). Error bars indicate the maximum variation between double samples.

The concentration of Pᵢ was higher in the whole wheat flour dough starting at 4 μmol/g wet dough (approximately equal to mM), and ending after 60 min of leavening at 11.8 μmol/g dough, while in the white wheat flour the start and end concentrations were 1.7 μmol/g and 5.8 μmol/g, respectively. The initial concentrations of IP6 were 9 μmol/g and 3 μmol/g for whole-wheat flour and white wheat flour, respectively, decreasing to 4.8 and 0.4 μmol/g after 60 minutes of leavening. This demonstrates that phytases are active in the dough.

Most previous investigations in the context of yeast degrading IP6 refer to a “yeast phytase”, frequently distinguishing it from phosphatases, without identifying genes and enzymes. Nayini et al. (1984) The phytase of yeast. Lebensm. Wiss. u-Technol. 17, 24-26; Harland et al. (1989) Effects of phytase from three yeasts on phytate reduction in Norwegian whole wheat flour. Cereal Chem. 4, 357-358; Nair et al. (1991) Phytic acid content reduction in canola meal by various microorganisms in a solid state fermentation process. Acta Biotechnologica. 11, 211-218. Lambrechts et al. (1992) Utilization of phytate by some yeasts. Biotechnol. Lett. 14, 61-66 Attempts to assess the yeast contribution to the observed phytase activity during bread leavening has sometimes been performed in conditions such as starvation in combination with 50° C., Harland et al. (1989) Effects of phytase from three yeasts on phytate reduction in Norwegian whole wheat flour. Cereal Chem. 4, 357-358.

The yeast phytase activity in conditions suitable for yeast growth were studied and it was observed that, provided absence of Pᵢ, S. cerevisiae is well adapted to utilize extracellular IP6 as a phosphorous source. The rate of IP6 degradation was rapid and supplied the yeast metabolic machinery with phosphorous without a decrease in growth rate, that arose from the increased energy cost of synthesizing the degrading enzymes. The phosphate-induced repression of phytase activity indicated that the PHO gene family in S. cerevisiae is involved.

In low concentrations of inorganic phosphate S. cerevisiae is known to induce the synthesis of at least three phosphatases that are exported out through the cellular membrane. It appears that the enzymes work best in oligomeric organization, as studied by Shnyreva et al. Shnyreva et al. (1996) Biochemical properties and excretion behavior of repressible acid phosphatases with altered subunit composition. Microbiol. Res. 151, 291-300, composed of a specific ratio between Pho5p (86%), Pho10p and Pho11p (14% together). The so-called “yeast phytase” may be the concerted action of the components in this oligomeric enzyme.

However, when using pNPP as substrate Shnyreva et al., Shnyreva et al. (1996) Biochemical properties and excretion behavior of repressible acid phosphatases with altered subunit composition. Microbiol. Res. 151, 291-300 have shown that also homopolymers of the individual secretory acid phosphatases have significant and different activities. In addition to being present in the largest amount, Pho5p was found to exert the highest specific activity. Fifteen times higher activity was obtained for Pho5p expressed alone as compared with Pho10p and Pho11p, respectively.

The PHO5 deletion did not yield a less efficient strain with respect to IP6 degradation. Several explanations could fit the data: (i) PHO5 was not important for phytase activity or (ii) the remaining level of Pho10p and Pho11p, organized without Pho5p, was sufficient for unchanged phytase activity. It may be that (iii) PHO10 and PHO11 became unregulated in the absence of PHO5. In fact, the absence of PHO10 or PHO11 has been shown to increase the Pho5p level Shnyreva et al. (1996) Biochemical properties and excretion behavior of repressible acid phosphatases with altered subunit composition. Microbiol. Res. 151, 291-300 and hence, the opposite may also be true. A (iv) different as yet unidentified phytase enzyme may exist. The S. cerevisiae gene DIA3 for instance, encodes a protein largely related to Pho3, Pho5, Pho10 and Pho11 (see e.g. the YPD database). Dia3p may be extracellular.

Since the significance of the different secretory phosphatases in IP6 specific degradation is not known a strain was created lacking only PHO10 (YS10). Using strain YS10 it was demonstrated that PHO10 was also dispensable for yeast phytase activity, shown by intact growth- and IP6 degradation rate. Thus, neither of PHO3, PHO5 or PHOJO was essential for high phytase activity. However, in a triple mutant (YM10), a very small, but significant decrease in rate of IP6 degradation was observed in CBS and a pronounced decrease in YPD, demonstrating two things. First, Pho10p is an enzyme with phytase activity since a change was observed when PHO10 was the only difference and second, a strain with only Pho11p alone (or an unknown phytase) still has a remarkable phytase activity in defined medium. For all these deletion strains the growth rate remained unchanged, so the net phytase activity is comparable also if expressed as biomass specific phytase activity. This further means that phosphorous never became limiting, even in a strain lacking three secretory phosphatases during growth on IP6. It is interesting that the main secretory phosphatase (Pho5p) is dispensable and that a strain encoding only one of two so-called minor secretory phosphatases, (Pho11p), is alone able to keep almost unchanged phytase activity.

To assess the phytase potential of solely Pho5p, two strains were constructed which expressed almost exclusively PHO5 during growth in repressing medium. The data obtained using these strains show that PHO5 by itself encodes a protein with phytase activity and is therefore likely to participate when a wildtype strain degrades IP6. When growing YM-p5 and YS-p5 in de-repressing media, additional phytase activity was observed as compared with the corresponding strains lacking the plasmids (FIG. 6). This was not necessarily expected since the increased rate of IP6 degradation leads to increase in Pᵢ concentration which in turn have a repressing impact on the genomic phytase encoding genes. The data reveals that yeast phytase activity is not the action of one gene product. However, simply by overexpressing one single gene strains were obtained with improved net phytase activity under both repressing and de-repressing conditions.

Furthermore, the regulatory components pho80p and pho85p are known to phosphorylate pho4p in conditions of high medium Pi levels. The phosphorylated pho4p retrieves affinity for a transporter and leaves the nucleus instead of acting as a transcription activator for several PHO genes, including the secretory phosphatases. If one or both of pho80p and pho85p is missing its kinase activity on pho4p is lost and pho4p will together with pho2p remain active as transcriptional activators for the sectretory phosphatases. The resulting mutant constitutively expresses PHO5, PHO10 and PHO11. Since deletions of PHO80 or PHO85 both yielded very strong constitutive phytase activity the involvement of the PHO system was again demonstrated.

It has previously been shown that the contribution of yeast to the phytase activity observed during bread leavening is very small, and not sufficient for a significant increase in iron and zinc absorption, Harland et al. (1989) Effects of phytase from three yeasts on phytate reduction in Norwegian whole wheat flour. Cereal Chem. 4, 357-358; Türk et al. (1992) Phytate degradation during bread making: effect of phytase addition. J. Cereal Science. 15, 281-294; Türk et al. (1996) Reduction in the levels of phytate during wholemeal bread making; Effect of yeast and wheat phytases. J. Cereal Science. 23, 257-264. The IP6 level and mineral uptake in human intestine was, however, improved when adding commercial Aspergillus phytase to dough or wheat rolls, respectively, Türk et al. (1992) Phytate degradation during bread making: effect of phytase addition. J. Cereal Science. 15, 281-294; Türk et al. (1996) Reduction in the levels of phytate during wholemeal bread making; Effect of yeast and wheat phytases. J. Cereal Science. 23, 257-264. It has been shown that the phytase activity is Pᵢ repressed . The concentration of Pᵢ was shown to be in the millimolar range which is above the level known to induce repression, Yoshida et al. (1989) Function of the PHO regulatory genes for repressible acid phosphatase synthesis in Saccharomyces cerevisiae. Mol. Gen. Genet. 217, 40-46. (10 mM Pᵢ is often used as “high” and 0.2 mM as “low”; Ogawa et al. (2000) New components of a system for phosphate accumulation and polyphosphate metabolism in Saccharomyces cerevisiae revealed by genomic expression analysis. Mol. Biol. Cell. 11, 4309-4321). Hence, in the typical bread making situation the PHO genes encoding Pho5p, Pho10p and Pho11p are not very active and, as shown, phytase activity is not substantially expressed. Furthermore, it has been shown that the IP6 degradation in cereals must be extensive to improve the iron absorption, Brune et al. (1992) Human iron absorption from bread: Inhibiting effects of cereal fibre, phytate and inositol phosphates with different numbers of phosphate groups. J. Nutr. 122, 442-449. As little as 0.5 μmol/g dry sample of IP6 and IP5 is inhibitory, Sandberg et al. (1999) Inositolphosphates with different number ofphosphate groups influence iron absorption in humans. Am. J. Clin. Nutr. 70, 240-246. The data on the content of IP6 in dough were expressed per wet weight (i.e. the dry content is higher), and even at the end of the fermentation it exceeds iron inhibitory levels. The reduction in IP6 taking place during dough fermentation with commercial baker's strains has been shown to be almost exclusively due to plant phytases present in the flour. A constitutively expressing yeast strain is desirable.

It has further been demonstrated herein an apparent preference for IP6 over pNPP when these were given together. In Nayaini et al., Nayini et al. (1984) The phytase of yeast. Lebensm. Wiss. u-Technol. 17, 24-26. yeast phytase and phosphatase were purified from a total biomass extraction of a Baker's yeast cake. The distinction between phytase (which is a phosphatase, for which one of the substrates is phytate) and phosphatase activity was that a phosphatase was able to hydrolyze alpha-glycerophosphate but unable to hydrolyze IP6. The purified phytase was assessed for substrate specific activity using 11 different phosphorous sources. Phenyl-phosphate was one of these and the specific activity with this substrate was 40 times higher than for IP6. The data set forth above, shows virtually no degradation of pNPP until IP6 is depleted. Even at six times higher pNPP concentration the data indicate an inhibition of pNPP hydrolysis by IP6. Although the yeast phytase is not one single enzyme the activity was shown to be very precise in regard to the position on the inositol ring to be hydrolyzed, consistent with previous data on one commercial baker's strain, Türk et al. (2000) Inositol hexaphosphate hydrolysis by baker's yeast. Capacity, kinetics, and degradation products. J. Agric. Food Chem. 48, 100-104. This specificity, the so-called 3-phytase activity, was true for all yeasts tested, including such different wild isolates of S. cerevisiae including those from fish intestine and clinical isolates of human pathogens, as well as for all deletion strains. Accordingly, the modified strains can be used to efficiently produce highly specific isomers of lower inositol phosphates.

In addition to the Saccharomyces phytase work we are screening non-Saccharomyces yeasts for the possibility of finding novel enzymes with desirable properties. Tested species are primarily from tropical- and cactus origin. Desired properties are high temperature optimum and/or a specificity yielding different isomers as compared with S. cerevisiae. So far, all tested species and strains have shown phytase activity. However, biochemical properties of the different enzymes have not yet been examined.

In addition to S. cerevisiae, other yeast species that are capable of expressing phytase can be transformed to exhibit increased phytase activity.

Although the present invention has been shown and described with respect to several preferred embodiments thereof, various changes, omissions and additions to the form and detail thereof, may be made therein, without departing from the spirit and scope of the invention.

What is claimed is:

1. A process for preparing a Saccharomyces cerevisiae yeast strain having improved phytase activity comprising:
modifying the strain by inserting the gene into the strain, and/or by deleting the regulatory, repressor gene PHO85 from the strain, whereby the insertion is either chromosomal or by transforming the strain using a plasmid containing the one or more of the genes, the strain overexpressing one or more of the genes.

2. (canceled)

3. The process according to claim 1 wherein the strain overexpresses PHO4.

4-9. (canceled)

10. The process according to claim 1 wherein PHO85 has been deleted to form a mutant pho85.

11. A modified strain of Saccharomyces cerevisiae that overexpresses the gene PHO4, and deletion of PHO85, the strain having increased phytase activity.

12-14. (canceled)

15. The modified Saccharomyces cerevisiae according to claim 11 wherein the strain overexpresses PHO4.

16-19. (canceled)

20. The modified Saccharomyces cerevisiae according to claim 11 wherein the regulating repressor gene PHO85 has been deleted from the strain to form a mutant pho85.

21. A fermented product having been fermented using a Saccharomyces cerevisiae according to claim 11, the product having a reduced content of inositol hexaphosphate.

22. The fermented product according to claim 22 wherein the product is a bakery product.

23. The fermented product according to claim 22 wherein the product is a gruel product.

24. The fermented product according to claim 22 wherein the product is a brewed product.

25. A method for the preparation of phytase comprising: growing one or more of the strains according to claim 11 in a growth medium to extracellularly produce phytase; and isolating the phytase.

26. A foodstuff comprising the phytase produced and isolated in accordance with claim 25.

27. A feedstuff comprising the phytase produced and isolated in accordance with claim 25.

28. A method for preparing specific isomers of inositiol phosphates and myoinositol by fermenting one or more of the strains according to claim 11.

29. The method according to claim 28 wherein I(1,2,4,5,6)P5 is produced and isolated.

30. The method according to claim 28 wherein I(1,2,5,6)P4 is produced and isolated.

31. The method according to claim 28 wherein I(1,2,6)P3 is produced and isolated.

32. The method for preparing specific isomers of inositiol phosphates and myoinositol, wherein I(1,2,)P2 is produced and isolated.

33. The method according to claim 28 wherein I(2)P1 is produced and isolated.

34. The method according to claim 28 wherein myo-inositiol is produced and isolated.

35. The use of one or more of the strains according to claim 11 as a live probiotic additive to foodstuff and feedstuff to obtain phytase activity in the gastrointestinal tract.

36. A modified strain of Saccharomyces cereviaisae according to claim 11 wherein the strain contains a chromosomal integration of the gene and/or genes or is transformed by a plasmid carrying the gene and/or genes.

37. A modified strain of Saccharomyces cereviaisae exhibiting increased phytase activity, the strain having being deposited under DMZ accession number DSM-No. 14929.

38. A method of producing phytase activity including providing a modified Saccharomyces cerevisiae, containing an overexpression of the gene PHO4, and deletion of PHO85.

39. A method for preparing inositol I(1,2)P2 by fermenting a Saccharomyces cerevisiae strain that overexpresses the gene PHO4 and lacks the regulatory, repressor gene pho85.


SEQUENCE LISTING (12 sequences)

SEQ ID NO: 1 - DNA, 31 bp, Artificial Sequence
  Description of Artificial Sequence Primer
cggaattcatgtttaaatctgttgtttattc

SEQ ID NO: 2 - DNA, 29 bp, Artificial Sequence
  Description of Artificial Sequence Primer
cggtcagataactctgttatcgagctcgc

SEQ ID NO: 3 - DNA, 9715 bp, Artificial Sequence
  Description of Artificial Sequence Synthetic plasmid pYX212 nucleotide sequence
gaattcatgtttaaatctgttgtttattcaattttagccgcttctttggccaatgcaggtaccattcccttaggcaaactagccgatgtcgacaagattggtacccaaaaagatatcttcccatttttgggtggtgccggaccatactactctttccctggcgactatggtatttctcgtgatttgcctgaaggttgtgaaatgaagcaactgcaaatggttggtagacatggtgaaagataccctactgtcagtctggctaagactatcaagagtacatggtataagttgagcaattacactcgtcaattcaacggctcattgtcattcttgaacgatgattacgagtttttcatccgtgatgacgatgatttggaaatggaaaccacttttgccaactcggacgatgttttgaacccatacactggtgaaatgaacgccaagagacatgctcgtgacttcttggctcaatacggttacatggtcgaaaaccaaaccagtttcgccgtttttacctctaattctaagagatgtcatgacactgctcaatatttcattgatggtttaggtgaccaattcaacatcaccttgcagactgtcagtgaagctgaatccgctggtgccaacactttgagtgcttgtaactcatgtcctgcttgggactacgatgccaatgatgacattgtaaatgaatacgacacaacctacttggatgacattgccaagagattgaacaaggaaaacaagggtttgaacttgacctcaactgacgctagtactttattctcgtggtgtgcatttgaagtgaacgctaaaggttacagtgatgtctgtgatattttcaccaaggatgaattagtccattactcctactaccaagacttgcacacttattaccatgagggtccaggttacgacattatcaagtctgtcggttccaacttgttcaatgcctcagtcaaattattaaagcaaagtgagattcaagaccaaaaggtttggttgagttttacccacgataccgatatcctaaactttttgaccaccgctggtataattgacgacaaaaacaacttaactgccgaatacgttccattcatgggcaacactttccacagatcctggtacgttcctcaaggtgctcgtgtctacaccgaaaaattccaatgttctaacgacacctacgtcagatacgtcattaacgatgctgttgttccaattgaaacctgttccactggtccagggttctcttgtgaaatcaatgacttctacgactatgctgaaaagagagtagccggtactgacttcctaaaggtctgtaacgtcagcagcgtcagtaactctactgaattgaccttctactgggactggaacactactcattacaacgccagtctattgagacaatagcccgggtatccgtatgatgtgcctgactacgcatgatatctcgagctcagctagctaactgaataaggaacaatgaacgtttttcctttctcttgttcctagtattaatgactgaccgatacatccctttttttttttgtctttgtctagctccagcttttgttccctttagtgagggttaattcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtggtttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctgtaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctggccccagcacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatcgaaaaatcagtcaatatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtgcggccgctctagaactagtggatcaattccacggactatagactatactagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctgcggccgcactctcactagtacgtcaggtggcacttttcggggaaatgtgcgcggaacccctatttgtttatttttctaaatacattcaaatatgtatccgctcatgagacaataaccgtgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcgctcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttggacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccggtcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcgaaatacgactcactatagggcgaattgggtaccgggccggccgtcgagcttgatggcatcgtggtgtcacgctcgtcgtttggtatggcttcattcagctccggttcccaacgatcaaggcgagttacatgatcccccatgttgtgaaaaaaagcggttagctcttcggtcctccgatcgttgtcagaagtaagttggccgcagtgttatcactcatggttatggcaggaactgcataattctcttactgtcatgccatccgtaagatgcttttctgtgactggtgtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcgatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggggatctacgtatggtcattcttcttcagattccctcatggagaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggacctcaatacattcagacacttctgacggtatcaccctacttattcccttcgagattatatctaggaacccatcaggttggtggaagattacccgttctaagacttttcagcttcctctattgatgttacactcggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacagg

SEQ ID NO: 4 - DNA, 60 bp, Artificial Sequence
  Description of Artificial Sequence Primer
cgatagattcaagctcagtttcgccttggttgtaaagtaggcagctgaagcttcgtacgc

SEQ ID NO: 5 - DNA, 61 bp, Artificial Sequence
  Description of Artificial Sequence Primer
ggtctatttactgttttaataaagtgtcgttgtagtgcttggggcagatgatgtcgaggcg

SEQ ID NO: 6 - DNA, 1313 bp, Artificial Sequence
  Description of Artificial Sequence Synthetic nucleotide sequence used for deletion of PHO10
cgatagattcaagctcagtttcgccttggttgtaaagtagccagctgaagcttcgtacgctgcaggtcgacggatccccgggttaattaaggcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtgagaaatataaaaggttaggatttgccactgaggttcttctttcatatacttccttttaaatcttgctaggatacagttctcacatcacatccgaacataaacaaccatgggtaggagggcttttgtagaaagaaatacgaacgaaacgaaaatcagcgttgccatcgctttggacaaagctcccttacctgaagagtcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaagagtactgacaataaaaagattcttgttttcaagaacttgtcatttgtatagtttttttatattgtagttgttctattttaatcaaatgttagcgtgatttatattttttttcgcctcgacatcatctgccccaagcactacaacgacactttattaaaacagtaaatagacc

SEQ ID NO: 7 - DNA, 30 bp, Artificial Sequence
  Description of Artificial Sequence Primer
cggaattcatgggccgtacaacttctgagg

SEQ ID NO: 8 - DNA, 29 bp, Artificial Sequence
  Description of Artificial Sequence Primer
cgctcgagtcacgtgctcacgttctgcag

SEQ ID NO: 9 - DNA, 9210 bp, Artificial Sequence
  Description of Artificial Sequence Synthetic plasmid pYX212 nucleotide sequence
gaattcatgggccgtacaacttctgagggaatacacggttttgtggacgatctagagcccaagagcagcattcttgataaagtcggagactttatcaccgtaaacacgaaacggcatgatgggcgcgaggacttcaacgagcaaaacgacgagctgaacagtcaagagaaccacaacagcagtgagaatgggaacgagaatgaaaatgaacaagacagtctcgcgttggacgacctagaccgcgcctttgagctggtggaaggtatggatatggactggatgatgccctcgcatgcgcaccactccccagctacaactgctacaatcaagccgcggctattatattcgccgctaatacacacgcaaagtgcggttcccgtaaccatttcgccgaacttggtcgctactgctacttccaccacatccgctaacaaagtcactaaaaacaagagtaatagtagtccgtatttgaacaagcgcagaggtaaacccgggccggattcggccacttcgctgttcgaattgcccgacagcgttatcccaactccgaaaccgaaaccgaaaccaaagcaatatccgaaagttattctgccgtcgaacagcacaagacgcgtatcaccggtcacggccaagaccagcagcagcgcagaaggcgtggtcgtagcaagtgagtctcctgtaatcgcgccgcacggatcgagccattcgcggtcgctgagtaagcgacggtcatcgggcgcgctcgtggacgatgacaagcgcgaatcacacaagcatgcagagcaagcacggcgtaatcgattagcggtcgcgctgcacgaactggcgtctttaatccccgcggagtggaaacagcaaaatgtgtcggccgcgccgtccaaagcgaccaccgtggaggcggcctgccggtacatccgtcacctacagcagaacgtgagcacgtgactcgagctcagctagctaactgaataaggaacaatgaacgtttttcctttctcttgttcctagtattaatgactgaccgatacatccctttttttttttgtctttgtctagctccagcttttgttccctttagtgagggttaattcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtggtttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctgtaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctggccccagcacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatcgaaaaatcagtcaatatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtgcggccgctctagaactagtggatcaattccacggactatagactatactagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctgcggccgcactctcactagtacgtcaggtggcacttttcggggaaatgtgcgcggaacccctatttgtttatttttctaaatacattcaaatatgtatccgctcatgagacaataaccgtgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcgctcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttggacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccggtcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcgaaatacgactcactatagggcgaattgggtaccgggccggccgtcgagcttgatggcatcgtggtgtcacgctcgtcgtttggtatggcttcattcagctccggttcccaacgatcaaggcgagttacatgatcccccatgttgtgaaaaaaagcggttagctcttcggtcctccgatcgttgtcagaagtaagttggccgcagtgttatcactcatggttatggcaggaactgcataattctcttactgtcatgccatccgtaagatgcttttctgtgactggtgtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcgatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggggatctacgtatggtcattcttcttcagattccctcatggagaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggacctcaatacattcagacacttctgacggtatcaccctacttattcccttcgagattatatctaggaacccatcaggttggtggaagattacccgttctaagacttttcagcttcctctattgatgttacactcggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacag

SEQ ID NO: 10 - DNA, 61 bp, Artificial Sequence
  Description of Artificial Sequence Primer
cagcgtatattggctttcctttaatctaatgccccaagcccacatacgatttaggtgacac

SEQ ID NO: 11 - DNA, 63 bp, Artificial Sequence
  Description of Artificial Sequence Primer
ggagttctcaagctcatctcgaagtgttttctgtcgcttatgaatacgactcactatagggtg

SEQ ID NO: 12 - DNA, 1922 bp, Artificial Sequence
  Description of Artificial Sequence Synthetic nucleotide sequence used for deletion of PHO80
cagcgtatattggctttcctttaatctaatgccccaagcccacatacgatttaggtgacactatagaacgcggccgccagctgaagcttcgtacgctgcaggtcgacggatccccgggttaattaaggcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgatatcagatccactagtggcctatgcggccgcggatctgccggtctccctatagtgagtcgtattcataagcgacagaaaacacttcgagatgagcttgagaactcc